(19) United States
(12) Patent Application Publication
(10) Pub. No.: US 2024/0350613 A1
(43) Pub. Date: Oct. 24, 2024

(54) OLIGOMERIZATION PARTICLE FORMATION FUSION PROTEIN WITH IMMUNOGENIC ANTIGEN

(71) Applicant: VIROVAX LLC, Lawrence, KS (US)

(72) Inventor: Sunil Abraham David, Lawrence, KS (US)

(21) Appl. No.: 18/641,914

(22) Filed: Apr. 22, 2024

Related U.S. Application Data
(60) Provisional application No. 63/497,656, filed on Apr. 21, 2023.

(51) Int. Cl. A61K 39/12; A61K 39/00; A61K 39/145; A61P 31/14; A61P 31/16; C07K 14/005

(52) U.S. Cl. CPC A61K 39/12; A61K 39/145; A61P 31/14; A61P 31/16; C07K 14/005; A61K 2039/64; C07K 2319/40; C07K 2319/735; C12N 2760/16122; C12N 2760/16134; C12N 2770/24121; C12N 2770/24134

(57) ABSTRACT

A fusion protein can include: a series of polypeptide domains starting from an N-terminus or C-terminus, the series of polypeptide domains comprising in order: an oligomerization domain having an oligomerization amino acid sequence that oligomerizes with other polypeptides having the oligomerization amino acid sequence; a first spacer domain having a first spacer amino acid sequence of at least 6 amino acids; an antigenic domain having an antigenic amino acid sequence of an antigen that is immunogenic, wherein the antigen is from one of: Domain III of an envelope glycoprotein of a flavivirus; hemagglutinin of a H5 influenza virus, or receptor binding domain of a SARS-COV-2 virus; a second spacer domain having a second spacer amino acid sequence of at least 6 amino acids; and optionally a purification tag having a purification amino acid sequence that binds with a purification substrate.

### CROSS-REFERENCE TO RELATED APPLICATIONS

This patent application claims priority to U.S. Provisional Application No. 63/497,656 filed Apr. 21, 2023, which provisional is incorporated herein by specific reference in its entirety.

### U.S. GOVERNMENT RIGHTS

This invention was made with government support under Contract HHSN272201800049C awarded by the National Institutes of Health. The government has certain rights in the invention.

### SEQUENCE LISTING

The instant application contains a Sequence Listing which has been submitted via Patent Center and is hereby incorporated by reference in its entirety. Said.xml copy, created on Apr. 22, 2024 are named V409310005US02, and is 55,423 bytes in size.

### BACKGROUND

### Field

The present disclosure relates to fusion proteins, and use of such fusion proteins as antigens, such as in immunizations against the antigens.

### Description of Related Art

An antigen is a molecule, typically a protein or a polysaccharide, that is capable of triggering an immune response in an organism. These molecules are recognized by the immune system as foreign invaders, prompting the body to produce antibodies or activate specific immune cells to neutralize or eliminate them. Antigens can come from a variety of sources, including pathogens like bacteria, viruses, and parasites, as well as non-pathogenic substances such as pollen, dust, or certain foods. They possess specific molecular structures, known as epitopes, which interact with receptors on immune cells, initiating the complex cascade of events that constitutes the immune response. Understanding the nature and behavior of antigens is crucial in fields such as immunology, vaccine development, and diagnostics, as it underpins our ability to combat infectious diseases and maintain overall health.

Obtaining an antigen for a vaccine can be a challenging endeavor due to various factors. Identifying the specific antigen that will elicit a protective immune response against a particular pathogen requires extensive research and experimentation. Scientists must carefully study the pathogen's structure, life cycle, and interaction with the immune system to pinpoint the most effective target. Additionally, some pathogens mutate rapidly, making it difficult to find a stable antigen that remains consistent over time. Thus, there is a need for antigens for vaccines that can be used in immunizations.

### SUMMARY

In some embodiments, a fusion protein can include: a series of polypeptide domains starting from an N-terminus or C-terminus, the series of polypeptide domains comprising in order: an oligomerization domain having an oligomerization amino acid sequence that oligomerizes with other polypeptides having the oligomerization amino acid sequence; a first spacer domain having a first spacer amino acid sequence of at least 7 amino acids; an antigenic domain having an antigenic amino acid sequence of an antigen that is immunogenic, wherein the antigen is from one of: Domain III of an envelope glycoprotein of a flavivirus; hemagglutinin of a H5 influenza virus, or receptor binding domain of a SARS-COV-2 virus; a second spacer domain having a second spacer amino acid sequence of at least 6 amino acids; and optionally a purification tag having a purification amino acid sequence that binds with a purification substrate, thereby enabling purification. In some aspects, the fusion protein includes the purification tag. In some aspects, the fusion protein series of polypeptide domains start from the N-terminus. In some aspects, the fusion protein series of polypeptide domains start from the C-terminus.

In some embodiments, the oligomerization amino acid sequence includes SEQ ID NO: 1, with at least 95% identity thereof.

In some embodiments, the first spacer amino acid sequence or second spacer amino acid sequence independently includes SEQ ID NO: 2, SEQ ID NO: 49, or SEQ ID NO: 50, or at least 95% identity thereof.

In some embodiments, the purification tag includes the sequence of SEQ ID NO: 3 or at least 95% identity thereof.

In some embodiments, the fusion protein includes: SEQ ID NO: 4 or at least 95% identity thereof; SEQ ID NO: 5 or at least 95% identity thereof; SEQ ID NO: 7 or at least 95% identity thereof; SEQ ID NO: 8 or at least 95% identity thereof; SEQ ID NO: 10 or at least 95% identity thereof; SEQ ID NO: 11 or at least 95% identity thereof; SEQ ID NO: 13 or at least 95% identity thereof; SEQ ID NO: 14 or at least 95% identity thereof; SEQ ID NO: 16 or at least 95% identity thereof; SEQ ID NO: 17 or at least 95% identity thereof; SEQ ID NO: 19 or at least 95% identity thereof; SEQ ID NO: 20 or at least 95% identity thereof; SEQ ID NO: 22 or at least 95% identity thereof; SEQ ID NO: 23 or at least 95% identity thereof; SEQ ID NO: 25 or at least 95% identity thereof; SEQ ID NO: 26 or at least 95% identity thereof; SEQ ID NO: 28 or at least 95% identity thereof; SEQ ID NO: 29 or at least 95% identity thereof; SEQ ID NO: 31 or at least 95% identity thereof; SEQ ID NO: 32 or at least 95% identity thereof; SEQ ID NO: 34 or at least 95% identity thereof; SEQ ID NO: 35 or at least 95% identity thereof; SEQ ID NO: 37 or at least 95% identity thereof; SEQ ID NO: 38 or at least 95% identity thereof; SEQ ID NO: 40 or at least 95% identity thereof; SEQ ID NO: 41 or at least 95% identity thereof; SEQ ID NO: 43 or at least 95% identity thereof; SEQ ID NO: 44 or at least 95% identity thereof; SEQ ID NO: 46 or at least 95% identity thereof; or SEQ ID NO: 47 or at least 95% identity thereof.

In some embodiments, an immunological composition (e.g., vaccine) can include: a pharmaceutically acceptable carrier; and the particle having the oligomerized fusion proteins. The pharmaceutically acceptable carrier can be an aqueous composition that is suitable for administration to a subject, such as intramuscular injection. In some aspects, the immunological composition can include an adjuvant.

A method of immunizing a subject can include: providing the fusion protein of one of the embodiments (e.g., in oligomerized particle and/or vaccine); and administering the fusion protein to the subject in an amount sufficient to induce an immunological response against the fusion protein. In some aspects, the method can include administering an adjuvant with the fusion protein to the subject.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE FIGURES

The foregoing and following information as well as other features of this disclosure will become more fully apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings.

FIG. 1 includes a graph that shows the antibody titer versus serum dilution factor for a mixture of fusion proteins NP-H5-RG71A/RG8A (SEQ ID NO: 40)+NP-H5-VIET04 (SEQ ID NO: 43), and with two different adjuvants; high neutralizing titers were observed to GFP-expressing reporter virus (Influenza A, American Wigeon/SC/22-000345-001/2021 (Clade 2.3.3.4b), especially when adjuvanted with CQUIM-MA.

FIG. 2 includes a graph that shows the antibody titer versus serum dilution factor for a mixture of fusion proteins NP-H5-RG71A/RG8A (SEQ ID NO: 40)+NP-H5-VIET04 (SEQ ID NO: 43), and with two different adjuvants; high neutralizing titers were observed to GFP-expressing reporter virus (Influenza A, Vietnam/1203/2004, especially when adjuvanted with CQUIM-MA.

FIG. 3 includes a graph that shows the antibody titer versus serum dilution factor for fusion protein NP-WNV (SEQ ID NO: 34), and with different adjuvants; high neutralizing titers were observed to GFP-expressing reporter virus (West Nile Virus NY99), especially when adjuvanted with CQUIM-MA.

FIG. 4 includes a graph that shows the antibody titer versus serum dilution factor for fusion protein NP-POWV (SEQ ID NO: 7) and a mixture of fusion proteins NP-WNV (SEQ ID NO: 34)+NP-POWV (SEQ ID NO: 7), and with two different adjuvants; good neutralizing antibodies to live Powassan virus were observed, especially when adjuvanted with CQUIM-MA.

The elements and components in the figures can be arranged in accordance with at least one of the embodiments described herein, and which arrangement may be modified in accordance with the disclosure provided herein by one of ordinary skill in the art.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the figures, can be arranged, substituted, combined, separated, and designed in a wide variety of different configurations, all of which are explicitly contemplated herein.

Generally, the present technology includes a fusion protein and methods of making the same, and use of the fusion protein in immunization protocols. The fusion protein includes in sequence from the N-terminus: (1) an oligomerization domain; (2) a spacer sequence; (3) an antigenic sequence; (4) a spacer sequence; and (5) a purification tag. This order is defined as the “Forward Order”. Alternatively, the fusion protein could also include in sequence from the N-terminus: (1) a purification tag, (2) a spacer sequence, (3) an antigenic sequence, (4) a spacer sequence, and (5) an oligomerization domain. This order is defined as the “Reverse Order”. Thus, the oligomerization domain can be present either at the N terminus or at the C terminus of the fusion protein.

The oligomerization domain allows the antigen to oligomerize (e.g., non-covalently self-assemble) into large proteins that can grow to the size of nanoparticles. This greatly enhances the immunogenicity of the antigens. Immunization in several animal models indicate strong antibody responses that are capable of neutralizing the viruses. Oligomerization is a process in which individual fusion peptides non-covalently self-assemble to form a larger structure called an oligomer, which can be a particle. The exact number of fusion proteins in an oligomer particle can vary depending on the context and the specific molecule. Oligomerization can significantly impact the function, stability, and activity of a protein. Many proteins exist naturally as oligomers, including enzymes, receptors, and structural proteins.

The spacer sequences flanking the virus-derived antigen sequences allow epitope separation, and facilitate recognition by B lymphocytes of relevant, antigen-derived epitopes. The spacer sequences can be any amino acid sequence that is not an antigen or oligomerization domain, or purification tag. The spacer sequence can be at least 6 amino acids, or at least 7 amino acids, up to 25 amino acids.

The purification tag can be an EPEA ((glutamic acid-proline-glutamic acid-alanine), SEQ ID NO: 3) tag at the C-terminus (or N-terminus) that allows the purification of the antigens using affinity chromatography.

In some embodiments, the sequences of the fusion proteins provided herein can omit the EPEA (SEQ ID NO: 3) tag or other purification tag. Thus, the fusion protein sequences can be devoid of a purification tag.

In some embodiments, the antigen is a sequence from Domain III of the Envelope Glycoprotein of a virus, such as flaviviruses. Domain III of the envelope glycoprotein (E-glycoprotein) of flaviviruses is a structural and functional domain that plays a key role in the virus's life cycle and interaction with host cells. The E-glycoprotein is a major surface protein of flaviviruses such as dengue, Zika, West Nile, and yellow fever viruses. It is involved in processes such as viral attachment, entry, and fusion with the host cell membrane. Domain III of the E-glycoprotein is the C-terminal domain and is structurally distinct from the other domains (I and II).

In some embodiments, the antigen is a sequence from hemagglutinin from a virus, such as H5 Influenza virus. Hemagglutinins are proteins that possess a specific affinity for certain sugar molecules. Since carbohydrate units exist in most animal cell membranes, the hemagglutinins may attach to these receptor groups.

In some embodiments, the antigen is a sequence from the receptor binding domain of a virus, such as SARS-COV-2 virus. The receptor binding domain (RBD) of SARS-CoV-2 is a critical region within the spike glycoprotein of the virus. This domain is responsible for binding to the host cell receptor, angiotensin-converting enzyme 2 (ACE2), which facilitates viral entry into host cells. The RBD is located within the larger S1 subunit of the spike protein, which is the portion of the protein that interacts with the host cell.

In some embodiments, the fusion proteins can include:
1. An oligomerization domain (ferritin-like domain from Pseudomonadaceae bacteria), the sequence of which is:

(SEQ ID NO: 1)
MANEGYHEPIDELTDETRDMHRAIISLMEELEAVDWYNQRVDACKDEALK
AILAHNRDEEKEHAAMVLEWIRRKDPRFDKELKDYLFTEKPIAHK;

2. A flanking “spacer” with the sequence: GGGSGGG (SEQ ID NO: 2) or GGSGG (SEQ ID NO: 49) or GGGSGG (SEQ ID NO: 50), which immediately precedes and follows the viral antigenic sequence;
3. An antigenic domain having a sequence from Domain III of the envelope glycoprotein of a flavivirus, or from hemagglutinin from H5 Influenza viruses, or from the RBD of SARS-COV-2 virus; and
4. A “purification tag” at the N-terminus or the C-terminus with the sequence: EPEA (SEQ ID NO: 3).

### Example Fusion Proteins

### Tick-Borne Flavivirus Vaccine Fusion Proteins

### 1. Kyasanur Forest Disease Virus:

The fusion protein can include an antigenic domain sequence from the Kyasanur Forest Disease virus, which fusion protein is referred to as NP-KFDV. The NP-KFDV includes the oligomerization domain, first spacer domain and second spacer domain bounding the antigenic domain from the Kyasanur Forest Disease virus. The forward order of the NP-KFDV fusion protein includes:

(SEQ ID NO: 4)
MANEGYHEPIDELTDETRDMHRAIISLMEELEAVDWYNQRVDACKDEALK
AILAHNRDEEKEHAAMVLEWIRRKDPRFDKELKDYLFTEKPIAHKGGGSG
GGGMTYTVCEGSKFAWKRPPTDSGHDTVVMEVTYTGSKPCRIPVRAVAHG
EPNVNVASLITPNPSMETTGGGFVELQLPPGDNIIYVGELSHQWFQKGST
IGRVLGGSGGGEPEA.

Alternatively, the fusion protein having the Kyasanur Forest Disease virus antigenic domain can have the polypeptide domains arranged in the reverse order The reverse order of the polypeptide domains arranged in the NP-KFDV fusion protein (i.e., NP-KFDV Reverse Order) is:

(SEQ ID NO: 5)
EPEAGGGSGGGGMTYTVCEGSKFAWKRPPTDSGHDTVVMEVTYTGSKPCR
IPVRAVAHGEPNVNVASLITPNPSMETTGGGFVELQLPPGDNIIYVGELS
HQWFQKGSTIGRVLGGSGGGMANEGYHEPIDELTDETRDMHRAIISLMEE
LEAVDWYNQRVDACKDEALKAILAHNRDEEKEHAAMVLEWIRRKDPRFDK
ELKDYLFTEKPIAHK.

These fusion proteins include the oligomerization domain of SEQ ID NO: 1; two spacer domains of SEQ ID NO: 2 flanking the antigenic domain; and the affinity tag of SEQ ID NO: 3. However, the affinity tag may be optional or omitted. The antigenic domain of the Kyasanur Forest Disease virus is from the E-glycoprotein of Domain III is:

(SEQ ID NO: 6)
GMTYTVCEGSKFAWKRPPTDSGHDTVVMEVTYTGSKPCRIPVRAVAHGE
PNVNVASLITPNPSMETTGGGFVELQLPPGDNIIYVGELSHQWFQKGSTI
GRVL.

### 2. Powassan Virus

The fusion protein can include an antigenic domain sequence from the Powassan Virus, which fusion protein is referred to as NP-POWV. The NP-POWV includes the oligomerization domain, first spacer domain and second spacer domain bounding the antigenic domain from the Powassan virus. The forward order of the polypeptide domains arranged in the NP-POWV fusion protein includes:

(SEQ ID NO: 7)
MANEGYHEPIDELTDETRDMHRAIISLMEELEAVDWYNQRVDACKDEALK
AILAHNRDEEKEHAAMVLEWIRRKDPRFDKELKDYLFTEKPIAHKGGGSG
GGTTYSMCDKTKFKWKRVPVDSGHDTVVMEVSYTGSDKPCRIPVRAVAHG
VPTTNVAMLITPNPTIETSGGGFIEMQLPLGDNIIYVGDLSQQWFQKGST
IGRMFGGSGGGEPEA.

Alternatively, the fusion protein having the Powassan virus antigenic domain sequence can include the polypeptide domains arranged in the reverse order. The reverse order of the polypeptide domains arranged in the NP-POWV fusion protein (i.e., NP-POWV Reverse Order) is:

(SEQ ID NO: 8)
EPEAGGGSGGGTTYSMCDKTKFKWKRVPVDSGHDTVVMEVSYTGSDKPCR
RAVAHGVPTTNVAMLITPNPTIETSGGGFIEMQLPLGDNIIYVGDLSQQW
FQKGSTIGRMFGGSGGGMANEGYHEPIDELTDETRDMHRAIISLMEELEA
VDWYNQRVDACKDEALKAILAHNRDEEKEHAAMVLEWIRRKDPRFDKELK
DYLFTEKPIAHK.

These fusion proteins include the oligomerization domain of SEQ ID NO: 1; two spacer domains of SEQ ID NO: 2 flanking the antigenic domain; and the affinity tag of SEQ ID NO: 3. However, the affinity tag may be optional or omitted. The antigenic domain of the Powassan virus is from the E-glycoprotein of Domain III and is:

(SEQ ID NO: 9)
TTYSMCDKTKFKWKRVPVDSGHDTVVMEVSYTGSDKPCRIPVRAVAHGVP
TTNVAMLITPNPTIETSGGGFIEMQLPLGDNIIYVGDLSQQWFQKGSTIG
RMF.

### Mosquito-Borne Flavivirus Vaccine Fusion Proteins

### 1. Dengue Type 1 Viruses

The fusion protein can include an antigenic domain sequence from the Dengue Type 1 virus, which fusion protein is referred to as NP-DENV Type 1. The NP-DENV Type 1 includes the oligomerization domain, first spacer domain and second spacer domain bounding the antigenic domain from the Dengue Type 1 virus. The forward order of the polypeptide domains arranged in the NP-DENV Type 1 fusion protein includes:

(SEQ ID NO: 10)
MANEGYHEPIDELTDETRDMHRAIISLMEELEAVDWYNQRVDACKDEALK
AILAHNRDEEKEHAAMVLEWIRRKDPRFDKELKDYLFTEKPIAHKGGGSG
GGLKGVSYVMCTGSFKLEKEVAETQHGTVLVQVKYEGTDAPCKIPISTQD
EKGVTQNGRLITANPIVTDKEKPVNIETEPPFGESYIVIGAGEKALKLSW
FKGGSGGGEPEA.

Alternatively, the fusion protein having the Dengue Type 1 virus antigenic domain sequence can include the polypeptide regions in the reverse order. The reverse order of the polypeptide domains arranged in the NP-DENV Type 1 fusion protein (i.e., NP-DENV Type 1 Reverse Order) is:

(SEQ ID NO: 11)
EPEAGGGSGGGLKGVSYVMCTGSFKLEKEVAETQHGTVLVQVKYEGTDAP
CKIPISTQDEKGVTQNGRLITANPIVTDKEKPVNIETEPPFGESYIVIGA
GEKALKLSWFKGGSGGGMANEGYHEPIDELTDETRDMHRAIISLMEELEA
VDWYNQRVDACKDEALKAILAHNRDEEKEHAAMVLEWIRRKDPRFDKELK
DYLFTEKPIAHK.

These fusion proteins include the oligomerization domain of SEQ ID NO: 1; two spacer domains of SEQ ID NO: 2 flanking the antigenic domain; and the affinity tag of SEQ ID NO: 3. However, the affinity tag may be optional or omitted. The antigenic domain of the Dengue Virus Type 1 virus is from the E-glycoprotein of Domain III is:

(SEQ ID NO: 12)
LKGVSYVMCTGSFKLEKEVAETQHGTVLVQVKYEGTDAPCKIPISTQDEK
GVTQNGRLITANPIVTDKEKPVNIETEPPFGESYIVIGAGEKALKLSWF
K.

### 2. Dengue Type 2 Viruses (Canonical)

The fusion protein can include an antigenic domain sequence from the Dengue Type 2 virus, which fusion protein is referred to as NP-DENV Type 2 (Canonical). The NP-DENV Type 2 (Canonical) includes the oligomerization domain, first spacer domain and second spacer domain bounding the antigenic domain from the Dengue Type 2 virus. The forward order of the polypeptide domains arranged in the NP-DENV Type 2 (Canonical) fusion protein includes:

(SEQ ID NO: 13)
MANEGYHEPIDELTDETRDMHRAIISLMEELEAVDWYNQRVDACKDEALK
AILAHNRDEEKEHAAMVLEWIRRKDPRFDKELKDYLFTEKPIAHKGGGSG
GGQLKGMSYSMCTGKFKVVKEIAETQHGTIVVRVQYEGDGSPCKIPFEIM
DLEKRHVLGRLITVNPIVTEKDSPVNIEAEPPFGDSYIIIGVEPGQLKLS
WFKKGGSGGGEPEA.

Alternatively, the fusion protein having the Dengue Type 2 virus antigenic domain sequence can have the polypeptide domains arranged in the reverse order. The reverse order of the polypeptide domains arranged in the NP-DENV Type 2 (Canonical) fusion protein (i.e., NP-DENV Type 2 (Canonical) Reverse Order) is:

(SEQ ID NO: 14)
EPEAGGGSGGGQLKGMSYSMCTGKFKVVKEIAETQHGTIVVRVQYEGDGS
PCKIPFEIMDLEKRHVLGRLITVNPIVTEKDSPVNIEAEPPFGDSYIIIG
VEPGQLKLSWFKKGGSGGGMANEGYHEPIDELTDETRDMHRAIISLMEEL
EAVDWYNQRVDACKDEALKAILAHNRDEEKEHAAMVLEWIRRKDPRFDKE
LKDYLFTEKPIAHK.

These fusion proteins include the oligomerization domain of SEQ ID NO: 1; two spacer domains of SEQ ID NO: 2 flanking the antigenic domain; and the affinity tag of SEQ ID NO: 3. However, the affinity tag may be optional or omitted. The antigenic domain of the Dengue Virus Type 2 (Canonical) virus is from the E-glycoprotein of Domain III is:

(SEQ ID NO: 15)
QLKGMSYSMCTGKFKVVKEIAETQHGTIVVRVQYEGDGSPCKIPFEIMDL
EKRHVLGRLITVNPIVTEKDSPVNIEAEPPFGDSYIIIGVEPGQLKLSWF
KK.

### 3. Dengue Type 2 Viruses (S16803)

The fusion protein can include an antigenic domain sequence from the Dengue Type 2 virus, which fusion protein is referred to as NP-DENV Type 2 (S16803). The NP-DENV Type 2 (S16803) includes the oligomerization domain, first spacer domain and second spacer domain bounding the antigenic domain from the Dengue Type 2 virus. The forward order of the polypeptide domains arranged in the NP-DENV Type 2 (S16803) fusion protein includes:

(SEQ ID NO: 16)
MANEGYHEPIDELTDETRDMHRAIISLMEELEAVDWYNQRVDACKDEALK
AILAHNRDEEKEHAAMVLEWIRRKDPRFDKELKDYLFTEKPIAHKGGGSG
GGQLKGMSYSMCTGKFKVVKEIAETQHGTIVIRVQYEGDGSPCKIPFEIM
DLEKRHVLGRLITVNPIVTEKDSPVNIEAEPPFGDSYIIIGVDPGQLKLN
WFKKGGSGGGEPEA.

Alternatively, the fusion protein having the Dengue Type 2 virus antigenic sequence can include the polypeptide domains arranged in the reverse order. The reverse order of the polypeptide domains arranged in the NP-DENV Type 2 (S16803) fusion protein (i.e., NP-DENV Type 2 [S168031] Reverse Order) is:

(SEQ ID NO: 17)
EPEAGGGSGGGQLKGMSYSMCTGKFKVVKEIAETQHGTIVIRVQYEGDGS
PCKIPFEIMDLEKRHVLGRLITVNPIVTEKDSPVNIEAEPPFGDSYIIIG
VDPGQLKLNWFKKGGSGGGMANEGYHEPIDELTDETRDMHRAIISLMEEL
EAVDWYNQRVDACKDEALKAILAHNRDEEKEHAAMVLEWIRRKDPRFDKE
LKDYLFTEKPIAHK.

These fusion proteins include the oligomerization domain of SEQ ID NO: 1; two spacer domains of SEQ ID NO: 2 flanking the antigenic domain; and the affinity tag of SEQ ID NO: 3. However, the affinity tag may be optional or omitted. The antigenic domain of the Dengue Virus Type 2 (S16803) virus is from the E-glycoprotein of Domain III is:

(SEQ ID NO: 18)
QLKGMSYSMCTGKFKVVKEIAETQHGTIVIRVQYEGDGSPCKIPFEIMDL
EKRHVLGRLITVNPIVTEKDSPVNIEAEPPFGDSYIIIGVDPGQLKLNWF
KK.

### 4. Dengue Type 3 Viruses

The fusion protein can include an antigenic domain sequence from the Dengue Type 3 virus, which fusion protein is referred to as NP-DENV Type 3. The NP-DENV Type 3 includes the oligomerization domain, first spacer domain and second spacer domain bounding the antigenic domain from the Dengue Type 3 virus. The forward order of the polypeptide domains arranged in the NP-DENV Type 3 fusion protein includes:

(SEQ ID NO: 19)
MANEGYHEPIDELTDETRDMHRAIISLMEELEAVDWYNQRVDACKDEALK
AILAHNRDEEKEHAAMVLEWIRRKDPRFDKELKDYLFTEKPIAHKGGGSG
GGLKGMSYAMCTNTFVLKKEVSETQHGTILIKVEYKGEDAPCKIPFSTED
GQGKAHNGRLITANPVVTKKEEPVNIEAEPPFGESNIVIGIGDNALKINW
YKKGGSGGGEPEA.

Alternatively, the fusion protein having the Dengue Type 3 virus antigenic domain sequence can include the polypeptide domains arranged in the reverse order. The reverse order of the polypeptide domains arranged in the NP-DENV Type 3 fusion protein (i.e., NP-DENV Type 3 Reverse Order) is:

(SEQ ID NO: 20)
EPEAGGGSGGGLKGMSYAMCTNTFVLKKEVSETQHGTILIKVEYKGEDAP
CKIPFSTEDGQGKAHNGRLITANPVVTKKEEPVNIEAEPPFGESNIVIGI
GDNALKINWYKKGGSGGGMANEGYHEPIDELTDETRDMHRAIISLMEELE
AVDWYNQRVDACKDEALKAILAHNRDEEKEHAAMVLEWIRRKDPRFDKEL
KDYLFTEKPIAHK.

These fusion proteins include the oligomerization domain of SEQ ID NO: 1; two spacer domains of SEQ ID NO: 2 flanking the antigenic domain; and the affinity tag of SEQ ID NO: 3. However, the affinity tag may be optional or omitted. The antigenic domain of the Dengue Virus Type 3 virus is from the E-glycoprotein of Domain III is:

(SEQ ID NO: 21)
LKGMSYAMCTNTFVLKKEVSETQHGTILIKVEYKGEDAPCKIPFSTEDGQ
GKAHNGRLITANPVVTKKEEPVNIEAEPPFGESNIVIGIGDNALKINWYK
K.

### 5. Dengue Type 4 Viruses

The fusion protein can include an antigenic domain sequence from the Dengue Type 4 virus, which fusion protein is referred to as NP-DENV Type 4. The NP-DENV Type 4 includes the oligomerization domain, first spacer domain and second spacer domain bounding the antigenic domain from the Dengue Type 4 virus. The forward order of the polypeptide domains arranged in the NP-DENV Type 4 fusion protein includes:

(SEQ ID NO: 22)
MANEGYHEPIDELTDETRDMHRAIISLMEELEAVDWYNQRVDACKDEALK
AILAHNRDEEKEHAAMVLEWIRRKDPRFDKELKDYLFTEKPIAHKGGGSG
GGIKGMSYTMCSGKFSIDKEMAETQHGTTVVKVKYEGTGAPCKVPIEIRD
VNKEKVVGRIISSTPFAENTNSVTNIELEPPFGDSYIVIGVGDSALTLHW
FRGGSGGGEPEA.

Alternatively, the fusion protein having the Dengue Type 4 virus antigenic domain sequence can include the polypeptide domains arranged in the reverse order. The reverse order of the polypeptide domains arranged in the NP-DENV Type 4 fusion protein (i.e., NP-DENV Type 4 Reverse Order) is:

(SEQ ID NO: 23)
EPEAGGGSGGGIKGMSYTMCSGKFSIDKEMAETQHGTTVVKVKYEGTGAP
CKVPIEIRDVNKEKVVGRIISSTPFAENTNSVTNIELEPPFGDSYIVIGV
GDSALTLHWFRGGSGGGMANEGYHEPIDELTDETRDMHRAIISLMEELEA
VDWYNQRVDACKDEALKAILAHNRDEEKEHAAMVLEWIRRKDPRFDKELK
DYLFTEKPIAHK.

These fusion proteins include the oligomerization domain of SEQ ID NO: 1; two spacer domains of SEQ ID NO: 2 flanking the antigenic domain; and the affinity tag of SEQ ID NO: 3. However, the affinity tag may be optional or omitted. The antigenic domain of the Dengue Virus Type 4 virus is from the E-glycoprotein of Domain III is:

(SEQ ID NO: 24)
IKGMSYTMCSGKFSIDKEMAETQHGTTVVKVKYEGTGAPCKVPIEIRDVN
KEKVVGRIISSTPFAENTNSVTNIELEPPFGDSYIVIGVGDSALTLHWF
R.

### 6. Zika Virus

The fusion protein can include an antigenic domain sequence from the Zika virus, which fusion protein is referred to as NP-ZIKV. The NP-ZIKV includes the oligomerization domain, first spacer domain and second spacer domain bounding the antigenic domain from the Zika virus. The forward order of the polypeptide domains arranged in the NP-ZIKV fusion protein includes:

(SEQ ID NO: 25)
MANEGYHEPIDELTDETRDMHRAIISLMEELEAVDWYNQRVDACKDEALK
AILAHNRDEEKEHAAMVLEWIRRKDPRFDKELKDYLFTEKPIAHKGGGSG
GGRLKGVSYSLCTAAFTFTKIPAETLHGTVTVELQYAGTDGPCKVPAQMA
VDMQTLTPVGRLITANPVITESTENSKMMLELDPPFGDSYIVIGVGEKKI
THHWHRSGGSGGGEPEA.

Alternatively, the fusion protein having the Zika virus antigenic domain sequence can include the polypeptide domains arranged in the reverse order The reverse order of the polypeptide domains arranged in the NP-ZIKV fusion protein (i.e., NP-ZIKV Reverse Order) is:

(SEQ ID NO: 26)
EPEAGGGSGGGRLKGVSYSLCTAAFTFTKIPAETLHGTVTVELQYAGTDG
PCKVPAQMAVDMQTLTPVGRLITANPVITESTENSKMMLELDPPFGDSYI
VIGVGEKKITHHWHRSGGSGGGMANEGYHEPIDELTDETRDMHRAIISLM
EELEAVDWYNQRVDACKDEALKAILAHNRDEEKEHAAMVLEWIRRKDPRF
DKELKDYLFTEKPIAHK.

These fusion proteins include the oligomerization domain of SEQ ID NO: 1; two spacer domains of SEQ ID NO: 2 flanking the antigenic domain; and the affinity tag of SEQ ID NO: 3. However, the affinity tag may be optional or omitted. The antigenic domain of the Zika virus is from the E-glycoprotein of Domain III is:

(SEQ ID NO: 27)
RLKGVSYSLCTAAFTFTKIPAETLHGTVTVELQYAGTDGPCKVPAQMAVD
MQTLTPVGRLITANPVITESTENSKMMLELDPPFGDSYIVIGVGEKKITH
HWHRS.

### 7. Yellow Fever Virus

The fusion protein can include an antigenic domain sequence from the Yellow

Fever virus, which fusion protein is referred to as NP-YF. The NP-YF includes the oligomerization domain, first spacer domain and second spacer domain bounding the antigenic domain from the Yellow Fever virus. The forward order of the polypeptide domains arranged in the NP-YF fusion protein includes:

(SEQ ID NO: 28)
MANEGYHEPIDELTDETRDMHRAIISLMEELEAVDWYNQRVDACKDEALK
AILAHNRDEEKEHAAMVLEWIRRKDPRFDKELKDYLFTEKPIAHKGGGSG
GGLKGTSYKMCTDKMSFVKNPTDTGHGTVVMQVKVPKGAPCKIPVIVADD
LTAAINKGILVTVNPIASTNDDEVLIEVNPPFGDSYIIVGTGDSRLTYQW
HKEGSSIGKLFTQTMKGAERLAVMGDAAWDESSAGGFFTSVGKGIHTVFG
SAFQGLFGGLSWITKVIMGAVLIWVGINTRNMTMSMSMILVGVIMMFLSL
GVGAGGSGGGEPEA.

Alternatively, the fusion protein having the Yellow Fever virus antigenic domain sequence can include the polypeptide domains arranged in the reverse order. The reverse order of the polypeptide domains arranged in the NP-YF fusion protein (i.e., NP-YF Reverse Order) is:

(SEQ ID NO: 29)
EPEAGGGSGGGLKGTSYKMCTDKMSFVKNPTDTGHGTVVMQVKVPKGAPC
KIPVIVADDLTAAINKGILVTVNPIASTNDDEVLIEVNPPFGDSYIIVGT
GDSRLTYQWHKEGSSIGKLFTQTMKGAERLAVMGDAAWDFSSAGGFFTSV
GKGIHTVFGSAFQGLFGGLSWITKVIMGAVLIWVGINTRNMTMSMSMILV
GVIMMFLSLGVGAGGSGGGMANEGYHEPIDELTDETRDMHRAIISLMEEL
EAVDWYNQRVDACKDEALKAILAHNRDEEKEHAAMVLEWIRRKDPRFDKE
LKDYLFTEKPIAHK.

These fusion proteins include the oligomerization domain of SEQ ID NO: 1; two spacer domains of SEQ ID NO: 2 flanking the antigenic domain; and the affinity tag of SEQ ID NO: 3. However, the affinity tag may be optional or omitted. The antigenic domain of the Yellow Fever virus is from the E-glycoprotein of Domain III is:

(SEQ ID NO: 30)
LKGTSYKMCTDKMSFVKNPTDTGHGTVVMQVKVPKGAPCKIPVIVADDLT
AAINKGILVTVNPIASTNDDEVLIEVNPPFGDSYIIVGTGDSRLTYQWHK
EGSSIGKLFTQTMKGAERLAVMGDAAWDESSAGGFFTSVGKGIHTVFGSA
FQGLFGGLSWITKVIMGAVLIWVGINTRNMTMSMSMILVGVIMMFLSLGV
GA.

### 8. Japanese B Encephalitis Virus

The fusion protein can include an antigenic domain sequence from the Japanese B Encephalitis virus, which fusion protein is referred to as NP-JEV. The NP-JEV includes the oligomerization domain, first spacer domain and second spacer domain bounding the antigenic domain from the Japanese B Encephalitis virus. The forward order of the polypeptide domains arranged in the NP-JEV fusion protein includes:

(SEQ ID NO: 31)
MANEGYHEPIDELTDETRDMHRAIISLMEELEAVDWYNQRVDACKDEALK
AILAHNRDEEKEHAAMVLEWIRRKDPRFDKELKDYLFTEKPIAHKGGGSG
GGLKGTTYGMCTEKFSFAKNPADTGHGTVVIELSYSGSDGPCKIPIVSVA
SLNDMTPVGRLVTVNPFVATSSANSKVLVEMEPPFGDSYIVVGMGDKQIN
HHWHKAGSTGGSGGGEPEA.

Alternatively, the fusion protein having the Japanese B Encephalitis virus antigenic domain sequence can include the polypeptide domains arranged in the reverse order. The reverse order of the polypeptide domains arranged in the NP-JEV fusion protein (i.e., NP-JEV Reverse Order) is:

(SEQ ID NO: 32)
EPEAGGGSGGGLKGTTYGMCTEKFSFAKNPADTGHGTVVIELSYSGSDGP
CKIPIVSVASLNDMTPVGRLVTVNPFVATSSANSKVLVEMEPPFGDSYIV
VGMGDKQINHHWHKAGSTGGSGGGMANEGYHEPIDELTDETRDMHRAIIS
LMEELEAVDWYNQRVDACKDEALKAILAHNRDEEKEHAAMVLEWIRRKDP
RFDKELKDYLFTEKPIAHK.

These fusion proteins include the oligomerization domain of SEQ ID NO: 1; two spacer domains of SEQ ID NO: 2 flanking the antigenic domain; and the affinity tag of SEQ ID NO: 3. However, the affinity tag may be optional or omitted. The antigenic domain of the Japanese B Encephalitis virus is from the E-glycoprotein of Domain III is:

(SEQ ID NO: 33)
LKGTTYGMCTEKFSFAKNPADTGHGTVVIELSYSGSDGPCKIPIVSVASL
NDMTPVGRLVTVNPFVATSSANSKVLVEMEPPFGDSYIVVGMGDKQINHH
WHKAGST.

### 9. West Nile Virus

The fusion protein can include an antigenic domain sequence from the West Nile virus, which fusion protein is referred to as NP-WNV. The NP-WNV includes the oligomerization domain, first spacer domain and second spacer domain bounding the antigenic domain from the West Nile virus. The forward order of the polypeptide domains arranged in the NP-WNV fusion protein includes:

(SEQ ID NO: 34)
MANEGYHEPIDELTDETRDMHRAIISLMEELEAVDWYNQRVDACKDEALK
AILAHNRDEEKEHAAMVLEWIRRKDPRFDKELKDYLFTEKPIAHKGGGSG
GGTTYGVCSKAFKFAGTPADTGHGTVVLELQYTGTDGPCKVPISSVASLN
DLTPVGRLVTVNPFVSVATANSKVLIELEPPFGDSYIVVGRGEQQINHHW
HKSGSSIGKAFTTTLKGAGGSGGGEPEA.

Alternatively, the fusion protein having the West Nile virus antigenic domain sequence can included the polypeptide domains arranged in the reverse order. The reverse order of the polypeptide domains arranged in the NP-WNV fusion protein (i.e., NP-WNV Reverse Order) is:

(SEQ ID NO: 35)
EPEAGGGSGGGTTYGVCSKAFKFAGTPADTGHGTVVLELQYTGTDGPCKV
PISSVASLNDLTPVGRLVTVNPFVSVATANSKVLIELEPPFGDSYIVVGR
GEQQINHHWHKSGSSIGKAFTTTLKGAGGSGGGMANEGYHEPIDELTDET
RDMHRAIISLMEELEAVDWYNQRVDACKDEALKAILAHNRDEEKEHAAMV
LEWIRRKDPREDKELKDYLFTEKPIAHK.

These fusion proteins include the oligomerization domain of SEQ ID NO: 1; two spacer domains of SEQ ID NO: 2 flanking the antigenic domain; and the affinity tag of SEQ ID NO: 3. However, the affinity tag may be optional or omitted. The antigenic domain of the West Nile virus is from the E-glycoprotein of Domain III is:

(SEQ ID NO: 36)
TTYGVCSKAFKFAGTPADTGHGTVVLELQYTGTDGPCKVPISSVASLNDL
TPVGRLVTVNPFVSVATANSKVLIELEPPFGDSYIVVGRGEQQINHHWHK
SGSSIGKAFTTTLKGA.

### 10. Murray Valley Encephalitis Virus

The fusion protein can include an antigenic domain sequence from the Murray Valley Encephalitis virus, which fusion protein is referred to as NP-MVEV. The NP-MVEV includes the oligomerization domain, first spacer domain and second spacer domain bounding the antigenic domain from the Murray Valley Encephalitis virus. The forward order of the polypeptide domains arranged in the NP-MVEV fusion protein includes:

(SEQ ID NO: 37)
MANEGYHEPIDELTDETRDMHRAIISLMEELEAVDWYNQRVDACKDEALK
AILAHNRDEEKEHAAMVLEWIRRKDPRFDKELKDYLFTEKPIAHKGGGSG
GGTTYGMCTEKFTFSKNPADTGHGTVVLELQYTGSDGPCKIPISSVASLN
DMTPVGRMVTANPYVASSTANAKVLVEIEPPFGDSYIVVGRGDKQINHHW
HKEGSSIGKAFSTTLKGAGGSGGGEPEA.

Alternatively, the fusion protein having the Murray Valley Encephalitis virus antigenic domain sequence can include the polypeptide domains arranged in the reverse order. The reverse order of the polypeptide domains arranged in the NP-MVEV fusion protein (i.e., NP-MVEV Reverse Order) is:

(SEQ ID NO: 38)
EPEAGGGSGGGTTYGMCTEKFTFSKNPADTGHGTVVLELQYTGSDGPCKI
PISSVASLNDMTPVGRMVTANPYVASSTANAKVLVEIEPPFGDSYIVVGR
GDKQINHHWHKEGSSIGKAFSTTLKGAGGSGGGMANEGYHEPIDELTDET
RDMHRAIISLMEELEAVDWYNQRVDACKDEALKAILAHNRDEEKEHAAMV
LEWIRRKDPREDKELKDYLFTEKPIAHK.

These fusion proteins include the oligomerization domain of SEQ ID NO: 1; two spacer domains of SEQ ID NO: 2 flanking the antigenic domain; and the affinity tag of SEQ ID NO: 3. However, the affinity tag may be optional or omitted. The antigenic domain of the Murray Valley Encephalitis virus is from the E-glycoprotein of Domain III is:

(SEQ ID NO: 39)
TTYGMCTEKFTFSKNPADTGHGTVVLELQYTGSDGPCKIPISSVASLNDM
TPVGRMVTANPYVASSTANAKVLVEIEPPFGDSYIVVGRGDKQINHHWHK
EGSSIGKAFSTTLKGA.

### Influenza Virus [H5N1] Vaccine Antigens

### 1. H5 Influenza (Clade 2.3.3.4b) Virus-Based H5-NP Antigen

The fusion protein can include an antigenic domain sequence from the Influenza virus [H5N1], which fusion protein is referred to as NP-H5-RG71A/RG8A. The NP-H5-RG71A/RG8A includes the oligomerization domain, first spacer domain and second spacer domain bounding the antigenic domain from the H5N1 Influenza virus. The forward order of the polypeptide domains arranged in the NP-H5-RG71A/RG8A fusion protein includes:

(SEQ ID NO: 40)
MANEGYHEEISDLSDETRDMHRAIVSLMEELEAVDWYNQRVDAAQDGDLK
AILAHNRDEEKEHAAMVLEWIRRKDPAFDKELKDYLFTEKPIAHSTGGGS
GGGPGSGDLNGVKPLILKDCSVAGWLLGNPMCDEFIRVPEWSYIVERANP
ANDLCYPGSLNDYEELKHLLSRINHFEKILIIPKSSWPNHETSLGVSAAC
PYQGAPSFFRNVVWLIKKNDAYPTIKISYNNTNREDLLILWGIHHSNNAE
EQTNLYKNPTTYISVGTSTLNQRLVPKIATRSQVNGQRGRMDFFWTILKP
DDAIHFESNGNFIAPEYAYKIVKKGDSTIMKSGVEYGGSGGGEPEA.

Alternatively, the fusion protein having the H5N1 Influenza virus antigenic domain sequence can include the polypeptide domains arranged in the reverse order. The reverse order of the polypeptide domains arranged in the NP-H5-RG71A/RG8A fusion protein (i.e., NP-H5-RG71A/RG8A Reverse Order) is:

(SEQ ID NO: 41)
EPEAGGGSGGGPGSGDLNGVKPLILKDCSVAGWLLGNPMCDEFIRVPEWS
YIVERANPANDLCYPGSLNDYEELKHLLSRINHFEKILIIPKSSWPNHET
SLGVSAACPYQGAPSFFRNVVWLIKKNDAYPTIKISYNNTNREDLLILWG
IHHSNNAEEQTNLYKNPTTYISVGTSTLNQRLVPKIATRSQVNGQRGRMD
FFWTILKPDDAIHFESNGNFIAPEYAYKIVKKGDSTIMKSGVEYGGSGGG
MANEGYHEEISDLSDETRDMHRAIVSLMEELEAVDWYNQRVDAAQDGDLK
AILAHNRDEEKEHAAMVLEWIRRKDPAFDKELKDYLFTEKPIAHST.

These fusion proteins include the oligomerization domain of SEQ ID NO: 1; two spacer domains of SEQ ID NO: 2 flanking the antigenic domain; and the affinity tag of SEQ ID NO: 3. However, the affinity tag may be optional or omitted. The antigenic domain of the H5N1 Influenza virus (Type H5) is from the hemagglutinin region is:

(SEQ ID NO: 42)
DLNGVKPLILKDCSVAGWLLGNPMCDEFIRVPEWSYIVERANPANDLCYP
GSLNDYEELKHLLSRINHFEKILIIPKSSWPNHETSLGVSAACPYQGAPS
FFRNVVWLIKKNDAYPTIKISYNNTNREDLLILWGIHHSNNAEEQTNLYK
NPTTYISVGTSTLNQRLVPKIATRSQVNGQRGRMDFFWTILKPDDAIHFE
SNGNFIAPEYAYKIVKKGDSTIMKSGVEY.

### 2. H5 Vietnam Influenza (Clade 1) Virus-Based H5-NP Antigen

The fusion protein can include an antigenic domain sequence from the Influenza virus [H5N1] from Vietnam, which fusion protein is referred to as NP-H5-VIET04. The NP-H5-VIET04 includes the oligomerization domain, first spacer domain and second spacer domain bounding the antigenic domain from the H5N1 Influenza virus from Vietnam. The forward order of the polypeptide domains arranged in the NP-H5-VIET04 fusion protein includes:

(SEQ ID NO: 43)
MANEGYHEEISDLSDETRDMHRAIVSLMEELEAVDWYNQRVDAAQDGDLK
AILAHNRDEEKEHAAMVLEWIRRKDPAFDKELKDYLFTEKPIAHSTGGGS
GGGPGSGDLDGVKPLILRDCSVAGWLLGNPMCDEFINVPEWSYIVEKANP
VNDLCYPGDENDYEELKHLLSRINHFEKIQIIPKSSWSSHEASLGVSSAC
PYQGKSSFFRNVVWLIKKNSTYPTIKRSYNNTNQEDLLVLWGIHHPNDAA
EQTKLYQNPTTYISVGTSTLNQRLVPRIATRSKVNGQSGRMEFFWTILKP
NDAINFESNGNFIAPEYAYKIVKKGDSTIMKSELEYGGSGGGEPEA.

Alternatively, the fusion protein having the H5N1 Vietnam Influenza Virus antigenic domain sequence can include the polypeptide domains arranged in the reverse order. The reverse order of the polypeptide domains arranged in the NP-H5-VIET04 fusion protein (i.e., NP-H5-VIET04 Reverse Order) is:

(SEQ ID NO: 44)
MANEGYHEEISDLSDETRDMHRAIVSLMEELEAVDWYNQRVDAAQDGDLK
AILAHNRDEEKEHAAMVLEWIRRKDPAFDKELKDYLFTEKPIAHSTEPEA
GGGSGGGPGSGDLDGVKPLILRDCSVAGWLLGNPMCDEFINVPEWSYIVE
KANPVNDLCYPGDFNDYEELKHLLSRINHFEKIQIIPKSSWSSHEASLGV
SSACPYQGKSSFFRNVVWLIKKNSTYPTIKRSYNNTNQEDLLVLWGIHHP
NDAAEQTKLYQNPTTYISVGTSTLNQRLVPRIATRSKVNGQSGRMEFFWT
ILKPNDAINFESNGNFIAPEYAYKIVKKGDSTIMKSELEYGGSGGGEPE
A.

These fusion proteins include the oligomerization domain of SEQ ID NO: 1; two spacer domains of SEQ ID NO: 2 flanking the antigenic domain; and the affinity tag of SEQ ID NO: 3. However, the affinity tag may be optional or omitted. The antigenic domain of the H5N1 Vietnam Influenza virus (Type H5) is from the Hemagglutinin region is:

(SEQ ID NO: 45)
DLDGVKPLILRDCSVAGWLLGNPMCDEFINVPEWSYIVEKANPVNDLCYP
GDENDYEELKHLLSRINHFEKIQIIPKSSWSSHEASLGVSSACPYQGKSS
FFRNVVWLIKKNSTYPTIKRSYNNTNQEDLLVLWGIHHPNDAAEQTKLYQ
NPTTYISVGTSTLNQRLVPRIATRSKVNGQSGRMEFFWTILKPNDAINFE
SNGNFIAPEYAYKIVKKGDSTIMKSELEY.

### A “Universal” SARS-COV-2 RBD-Based Antigen

### 1. NP-SARS-COV-2 Antigen-Forward Order:

The fusion protein can include an antigenic domain sequence from the SARS-CoV-2 virus, which fusion protein is referred to as NP-SARS-COV-2. The NP-SARS-CoV-2 includes the oligomerization domain, first spacer domain and second spacer domain bounding the antigenic domain from the SARS-COV-2 virus. The forward order of the polypeptide domains arranged in the NP-SARS-COV-2 fusion protein includes:

(SEQ ID NO: 46)
MANEGYHEPIDELTDETRDMHRAIISLMEELEAVDWYNQRVDACKDEALK
AILAHNRDEEKEHAAMVLEWIRRKDPRFDKELKDYLFTEKPIAHKGGGSG
GGPGSGNITNLCPFPEVFNATPFASVYAWNRPRISNCVADYSVLYNPAPF
FTFKCYGVSPTKLNDLCFTNVYADSFVIRGPEVPQIAPGQTGPIADYNYK
LPDNFTGCVIAWNSNPLDSAPAGNYNYPYRLFRKSPLKPFERDISTEIYQ
AGPKPCNGVPGFNCYPPLQSYGFRPTYGVGHQPYRVVVLSFENGTNGTTV
CGPGSGGGEPEA.

Alternatively, the fusion protein having the SARS-COV-2 Virus antigenic domain sequence can include the polypeptide domains arranged in the reverse order. The reverse order of the polypeptide domains arranged in the NP-SARS-COV-2 fusion protein (i.e., NP-SARS-COV-2 Reverse Order) is:

(SEQ ID NO: 47)
EPEAGGGSGGGPGSGNITNLCPFPEVFNATPFASVYAWNRPRISNCVADY
SVLYNPAPFFTFKCYGVSPTKLNDLCFTNVYADSFVIRGPEVPQIAPGQT
GPIADYNYKLPDNFTGCVIAWNSNPLDSAPAGNYNYPYRLFRKSPLKPFE
RDISTEIYQAGPKPCNGVPGFNCYPPLQSYGFRPTYGVGHQPYRVVVLSF
ENGTNGTTVCGPGSGGGMANEGYHEPIDELTDETRDMHRAIISLMEELEA
VDWYNQRVDACKDEALKAILAHNRDEEKEHAAMVLEWIRRKDPRFDKELK
DYLFTEKPIAHK.

These fusion proteins include the oligomerization domain of SEQ ID NO: 1; two spacer domains of SEQ ID NO: 2 flanking the antigenic domain; and the affinity tag of SEQ ID NO: 3. However, the affinity tag may be optional or omitted. The antigenic domain of the SARS-COV-2 virus is from the receptor binding domain is:

(SEQ ID NO: 48)
NITNLCPFPEVFNATPFASVYAWNRPRISNCVADYSVLYNPAPFFTFKCY
GVSPTKLNDLCFTNVYADSFVIRGPEVPQIAPGQTGPIADYNYKLPDNFT
GCVIAWNSNPLDSAPAGNYNYPYRLFRKSPLKPFERDISTEIYQAGPKPC
NGVPGFNCYPPLQSYGFRPTYGVGHQPYRVVVLSFENGTNGTTVCGP.

Particular viral proteins of interest include: an antigen from a virus in the flaviviridae family (genus flavivirus), such as from Kyasanur Forest Disease virus, Powassan virus, Yellow Fever virus, Zika virus, Japanese B Encephalitis virus, four serotypes of Dengue viruses, Tick-borne Encephalitis virus, West Nile virus, Murray Valley Encephalitis virus. These can be tick-borne or mosquito-borne viruses. Also, the antigen can be from Influenza virus, such as H5N1. The antigen can also be a universal SARS-COV-2 antigen.

The antigen can be the full sequences as shown herein. That is, there can be variations in the sequence so long as the sequence functions as described herein. Similarly, the oligomerization regions, spacers, or affinity tags may have variations so long as they retain functionality. The sequences in the sequence listing may vary by having at least 99% identity, at least 98% identity, at least 95% identity, at least 90% identity, at least 85% identity, or at least 80% identity.

The composition of the invention will typically, in addition to the components mentioned above, comprise one or more ‘pharmaceutically acceptable carriers’, which include any carrier that does not itself induce the production of antibodies. Such carriers are well known. Suitable carriers can include typically large, slowly metabolized macromolecules such as proteins, polysaccharides, polylactic acids, polyglycolic acids, polymeric amino acids, amino acid copolymers, and lipid aggregates (such as oil droplets or liposomes). Such carriers are well known to those of ordinary skill in the art. The vaccines may also contain diluents, such as water, saline, glycerol, etc. Additionally, auxiliary substances, such as wetting or emulsifying agents, pH buffering substances, and the like, may be present.

Compositions of the invention may be administered in conjunction with immunoregulatory agents. In particular, compositions will usually include one or more adjuvants.

### EXAMPLES

### Example 1

Rabbits (n=4 per cohort) were immunized as follows: a prime dose on Day 0 and a single boost on Day 15. The antigen domain (20 micrograms/dose) used had the following H5 (Clade 2.3.3.4b) sequence: MANEGYHEEISDLSDETRDMHRAIVSLMEELEAVDWYNQRVDAAQDGDLKAIL AHNRDEEKEHAAMVLEWIRRKDPAFDKELKDYLFTEKPIAHSTGGGSGGGPGSG DLNGVKPLILKDCSVAGWLLGNPMCDEFIRVPEWSYIVERANPANDLCYPGSLN DYEELKHLLSRINHFEKILIIPKSSWPNHETSLGVSAACPYQGAPSFFRNVVWLIKK NDAYPTIKISYNNTNREDLLILWGIHHSNNAEEQTNLYKNPTTYISVGTSTLNQRL VPKIATRSQVNGQRGRMDFFWTILKPDDAIHFESNGNFIAPEYAYKIVKKGDSTI MKSGVEYGGSGGGEPEA (SEQ ID NO: 40). The adjuvants used were either CQUIM-MA, or Alhydroxiquim-II, both at 20 micrograms imidazoquinoline content/dose. The serum from the immunization (e.g., Immune-1 sera) were obtained on Day 25, and were examined for neutralizing antibodies using a commercially-available, non-replicating GFP-expressing reporter virus.

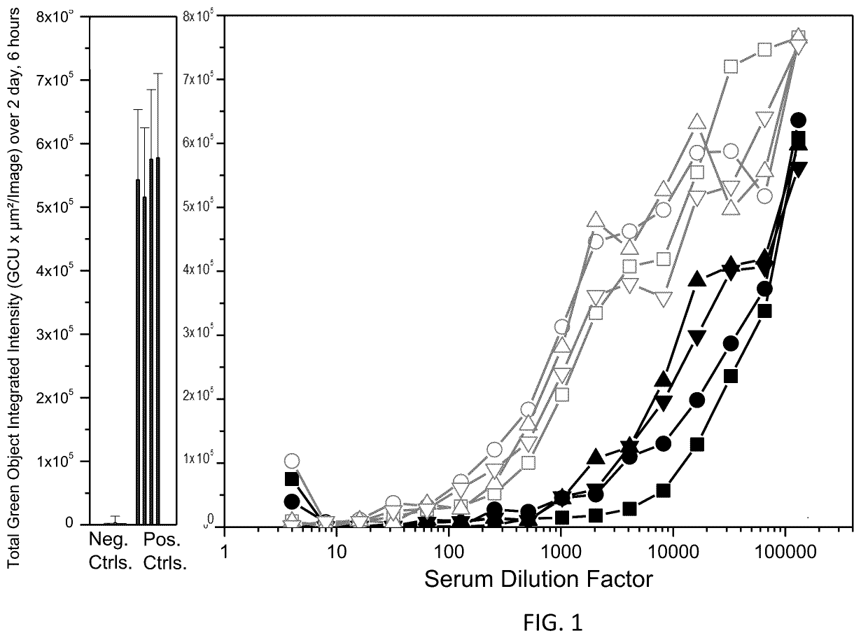

FIG. 1 shows the data for antibody titers for the Experiment VV-45, with H5 Antigens introduced to the rabbit with an intramuscular injection. The solid data points are for Experiment VV-45 Cohort A after Immunization-1, with fusion proteins NP-H5-RG71A/RG8A+NP-H5-VIET04, with the adjuvant being Cquim-1 (MA). The solid square data is for rabbit experiment 3.VV45.VVX133.Imm1. The solid circle data is for rabbit experiment 3.VV45.VVX134.Imm1. The solid up triangle is for rabbit experiment 3.VV44.VVX135.Imm1. The solid down triangle is for rabbit experiment 3.VV45.VVX136.Imm1. The accelerated regimen included: Day 0-prime; Day 15-boost 1; day 25-obtain Immune-1 sera.

The outline data points are for Experiment VV-45 Cohort B after Immunization-1, with fusion proteins NP-H5-RG71A/RG8A+NP-H5-VIET04, with the adjuvant being Alhydroxiquim-II. The outline square data is for rabbit experiment 4. VV45.VVX137.Imm1. The outline circle is for rabbit experiment 4.VV45.VVX138.Imm1. The outline up triangle is for rabbit experiment 4. VV45.VVX139.Imm1. The down triangle data is for rabbit experiment 4.VV45.VVX140.Imm1.

The data for the negative controls is minimal and the data for the positive controls matches the data from the immunization. Accordingly, these fusion proteins are shown to induce an immunological response.

### Example 2

Rabbits (n=4 per cohort) were immunized as follows: a prime dose on Day 0 and a single boost on Day 15. The antigen (20 micrograms/dose) used had the following H5 (Clade 1) sequence: MANEGYHEEISDLSDETRDMHRAIVSLMEELEAVDWYNQRVDAAQDGDLKAIL AHNRDEEKEHAAMVLEWIRRKDPAFDKELKDYLFTEKPIAHSTGGGSGGGPGSG DLDGVKPLILRDCSVAGWLLGNPMCDEFINVPEWSYIVEKANPVNDLCYPGDFN DYEELKHLLSRINHFEKIQIIPKSSWSSHEASLGVSSACPYQGKSSFFRNVVWLIKK NSTYPTIKRSYNNTNQEDLLVLWGIHHPNDAAEQTKLYQNPTTYISVGTSTLNQR LVPRIATRSKVNGQSGRMEFFWTILKPNDAINFESNGNFIAPEYAYKIVKKGDSTI MKSELEYGGSGGGEPEA (SEQ ID NO: 43). The adjuvants used were either CQUIM-MA, or Alhydroxiquim-II, both at 20 micrograms imidazoquinoline content/dose. The serum from the immunization (e.g., Immune-1 sera) were obtained on Day 25, and were examined for neutralizing antibodies using a commercially-available, non-replicating GFP-expressing reporter virus.

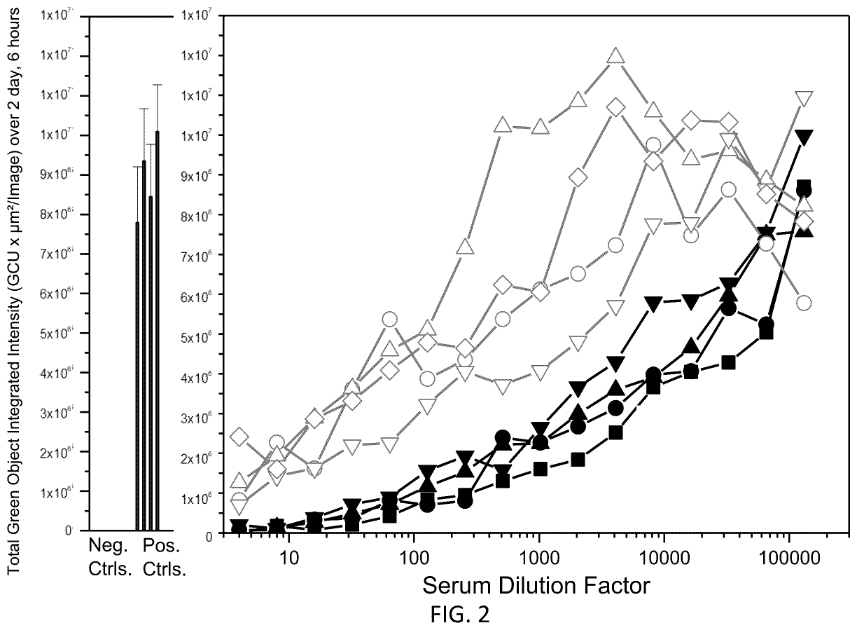

FIG. 2 shows the data for antibody titers for the Experiment VV-48, with H5 Antigens introduced to the rabbit with an intramuscular injection. The solid data points are for Experiment VV-48 Cohort A after Immunization-1, with fusion protein NP-H5-RG71A/RG8A+NP-H5-VIET04, with the adjuvant being Cquim-1 (MA). The accelerated regimen included: Day 0-prime; Day 15-boost 1; day 25-collect Immune-1 sera. The solid square data is for rabbit experiment 1.VV48.VVX177.Imm1. The solid circle data is for rabbit experiment 1.VV48.VVX178.Imm1. The solid up triangle is for rabbit experiment 1.VV48.VVX179.Imm1. The solid down triangle is for rabbit experiment 1.VV48.VVX180.Imm1.

The outline data points are for Experiment VV-48 with Cohort B after Immunization-1, with fusion proteins NP-H5-RG71A/RG8A+NP-H5-VIET04, with the adjuvant being Alhydroxiquim-II. The outline circle data is for rabbit experiment 4.VV48.VVX189.Imm1. The outline up triangle is for experiment 4.VV48.VVX190.Imm1. The outline down triangle is for rabbit experiment 4. VV48.VVX191.Imm1. The outline diamond data is for rabbit experiment 4.VV48.VVX192.Imm1.

The data for the negative controls is minimal and the data for the positive controls matches the data from the immunization. Accordingly, these fusion proteins are shown to induce an immunological response.

### Example 3

Rabbits (n=4 per cohort) were immunized as follows: a prime dose on Day 0 and a single boost on Day 15. The antigen (20 micrograms/dose) used had the following West Nile Virus sequence: MANEGYHEPIDELTDETRDMHRAIISLMEELEAVDWYNQRVDACKDEALKAILA HNRDEEKEHAAMVLEWIRRKDPRFDKELKDYLFTEKPIAHKGGGSGGGTTYGV CSKAFKFAGTPADTGHGTVVLELQYTGTDGPCKVPISSVASLNDLTPVGRLVTVN PFVSVATANSKVLIELEPPFGDSYIVVGRGEQQINHHWHKSGSSIGKAFTTTLKGA GGSGGGEPEA (SEQ ID NO: 34). The adjuvants used were either CQUIM-MA, or Alhydroxiquim-II, both at 20 micrograms imidazoquinoline content/dose. After the immunization, the serum (e.g., Immune-1 sera) were obtained on Day 25, and were examined for neutralizing antibodies using a commercially-available, non-replicating GFP-expressing reporter virus.

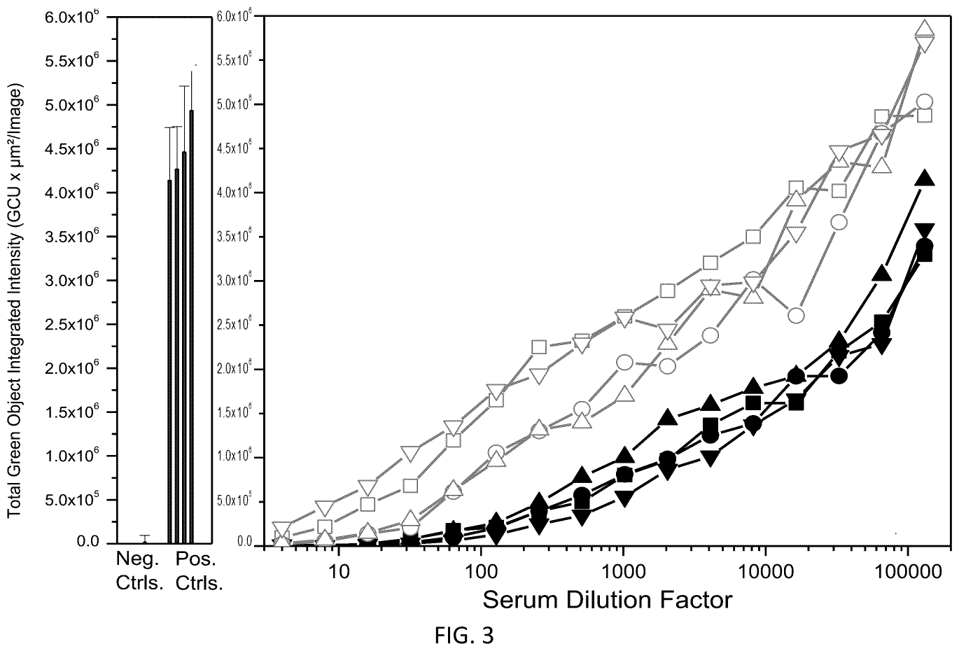

FIG. 3 shows the data for antibody titers for the Experiment VV-49, with NP-WNV introduced to each rabbit with a intramuscular injection. The solid data points are for Experiment VV-49 Cohort A after Immunization-1, with fusion protein NP-WNV, with the adjuvant being Cquim-1 (MA). The solid square data is for rabbit experiment 1.VV49.VVX193.Imm1. The solid circle data for rabbit is experiment 1.VV49.VVX194.Imm1. The solid up triangle is for rabbit experiment 1.VV49.VVX195.Imm1. The solid down triangle is for rabbit experiment 1.VV49.VVX196.Imm1.

The outline data points are for Experiment VV-49 Cohort B after Immunization-1, with fusion protein NP-WNV, with the adjuvant being Alhydroxiquim-II. The outline square data is for rabbit experiment 4.VV49.VVX205.Imm1. The outline circle data is for rabbit experiment 4.VV49.VVX205.Imm1. The outline up triangle is for rabbit experiment 4.VV49.VVX207.Imm1. The outline down triangle is for rabbit experiment 4.VV49.VVX208.Imm1.

The data for the negative controls is minimal and the data for the positive controls matches the data from the immunization. Accordingly, these fusion proteins are shown to induce an immunological response.

### Example 4

Rabbits (n=4 per cohort) were immunized as follows: a prime dose on Day 0 and two boosts on Day 15 and Day 30. The antigen (20 micrograms/dose) used for Cohort A had the following Powassan Virus sequence: MANEGYHEPIDELTDETRDMHRAIISLMEELEAVDWYNQRVDACKDEALKAILA HNRDEEKEHAAMVLEWIRRKDPRFDKELKDYLFTEKPIAHKGGGSGGGTTYSM CDKTKFKWKRVPVDSGHDTVVMEVSYTGSDKPCRIPVRAVAHGVPTTNVAMLI TPNPTIETSGGGFIEMQLPLGDNIIYVGDLSQQWFQKGSTIGRMFGGSGGGEPEA (SEQ ID NO: 7). Cohorts B and C received a mixture of the above-described Powassan Virus antigen plus a West Nile Virus antigen of the following sequence: MANEGYHEPIDELTDETRDMHRAIISLMEELEAVDWYNQRVDACKDEALKAILA HNRDEEKEHAAMVLEWIRRKDPRFDKELKDYLFTEKPIAHKGGGSGGGTTYGV CSKAFKFAGTPADTGHGTVVLELQYTGTDGPCKVPISSVASLNDLTPVGRLVTVN PFVSVATANSKVLIELEPPFGDSYIVVGRGEQQINHHWHKSGSSIGKAFTTTLKGA GGSGGGEPEA (SEQ ID NO: 34). Both antigens were at 20 micrograms per dose. The adjuvants used were either CQUIM-MA, or Alhydroxiquim-II, both at 20 micrograms imidazoquinoline content/dose. After immunization, the serum (e.g., Immune-2 sera) were obtained on Day 38, and were examined for neutralizing antibodies using live Powassan virus

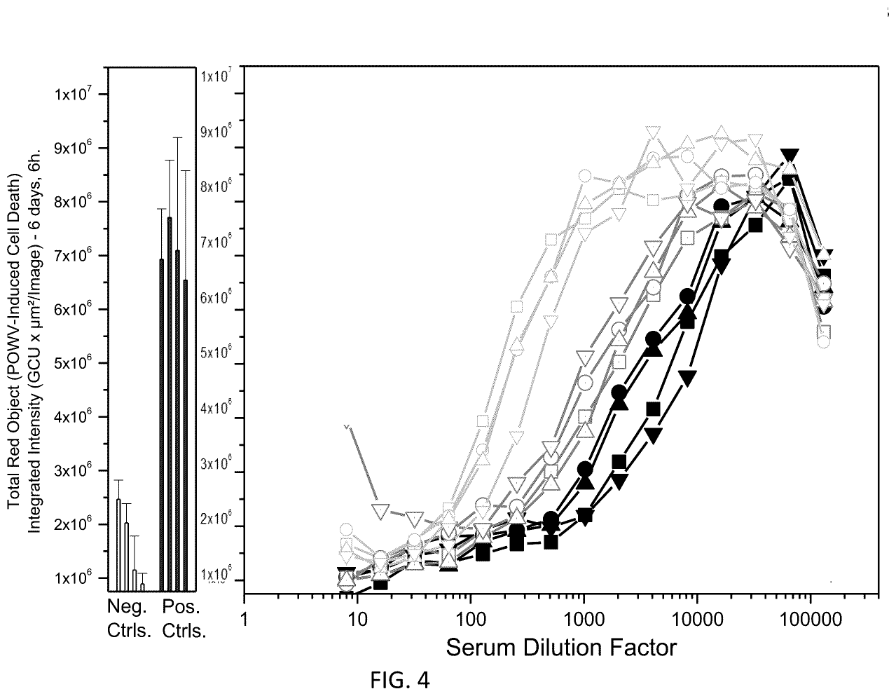

FIG. 4 shows the data for antibody titers for the Experiment VV-46, with NP-WNV and NP-POWV antigens introduced to each rabbit with a intramuscular injection. The solid data points are for Experiment VV-46 Cohort A after Immunization-2, with fusion protein NP-POW, with the adjuvant being Cquim-1 (MA). The solid square data is for rabbit experiment 2.VV46.VVX145.Imm2. The solid circle data is for rabbit experiment 2.VV46.VVX146.Imm2. The solid up triangle is for rabbit experiment 2.VV46.VVX147.Imm2. The solid down triangle is for rabbit experiment 2.VV46.VVX148.Imm2.

The outline data points are for Experiment VV-46 Cohort B after Immunization-2, with fusion proteins NP-WNV+NP-POWV, with the adjuvant being Cquim-1 (MA). The outline square data is for rabbit experiment 3.VV46.VVX149.Imm2. The outline circle data is for rabbit experiment 3.VV46.VVX150.Imm2. The outline up triangle is for rabbit experiment 3.VV46.VVX151.Imm2. The outline down triangle is for rabbit experiment 3.VV46.VVX152.Imm2.

The light outline data points are for Experiment VV-46 Cohort C after Immunization-2, with fusion proteins NP-WNV+NP-POWV, with the adjuvant being Alhydroxiquim-II. The light outline square data is for rabbit experiment 4.VV46.VVX153.Imm2. The light outline circle data is for rabbit experiment 4.VV46.VVX154.Imm2. The light outline up triangle is for rabbit experiment 4.VV46.VVX155.Imm2. The light outline down triangle is for rabbit experiment 4.VV46.VVX156.Imm2.

The data for the negative controls is minimal and the data for the positive controls matches the data from the immunization. Accordingly, these fusion proteins are shown to induce an immunological response.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

In addition, where features or aspects of the disclosure are described in terms of Markush groups, those skilled in the art will recognize that the disclosure is also thereby described in terms of any individual member or subgroup of members of the Markush group.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims.

1. A fusion protein comprising:
a series of polypeptide domains starting from an N-terminus or C-terminus, the series of polypeptide domains comprising in order:
an oligomerization domain having an oligomerization amino acid sequence that oligomerizes with other polypeptides having the oligomerization amino acid sequence;
a first spacer domain having a first spacer amino acid sequence of at least 6 amino acids;
an antigenic domain having an antigenic amino acid sequence of an antigen that is immunogenic, wherein the antigen is from one of: Domain III of an envelope glycoprotein of a flavivirus; hemagglutinin of a H5 influenza virus, or a receptor binding domain of a SARS-COV-2 virus;
a second spacer domain having a second spacer amino acid sequence of at least 6 amino acids; and
optionally a purification tag having a purification amino acid sequence that binds with a purification substrate.

2. The fusion protein of claim 1, wherein the oligomerization amino acid sequence includes SEQ ID NO: 1 or at least 95% identity thereof.

3. The fusion protein of claim 1, wherein the first spacer amino acid sequence or second spacer amino acid sequence independently includes SEQ ID NO: 2, SEQ ID NO: 49, or SEQ ID NO: 50, or at least 95% identity thereof.

4. The fusion protein of claim 1, comprising the purification tag having the purification amino acid sequence of SEQ ID NO: 3 or at least 95% identity thereof.

5. The fusion protein of claim 1, comprising:
SEQ ID NO: 4 or at least 95% identity thereof;
SEQ ID NO: 5 or at least 95% identity thereof;
SEQ ID NO: 7 or at least 95% identity thereof;
SEQ ID NO: 8 or at least 95% identity thereof;
SEQ ID NO: 10 or at least 95% identity thereof;
SEQ ID NO: 11 or at least 95% identity thereof;
SEQ ID NO: 13 or at least 95% identity thereof;
SEQ ID NO: 14 or at least 95% identity thereof;
SEQ ID NO: 16 or at least 95% identity thereof;
SEQ ID NO: 17 or at least 95% identity thereof;
SEQ ID NO: 19 or at least 95% identity thereof;
SEQ ID NO: 20 or at least 95% identity thereof;
SEQ ID NO: 22 or at least 95% identity thereof;
SEQ ID NO: 23 or at least 95% identity thereof;
SEQ ID NO: 25 or at least 95% identity thereof;
SEQ ID NO: 26 or at least 95% identity thereof;
SEQ ID NO: 28 or at least 95% identity thereof;
SEQ ID NO: 29 or at least 95% identity thereof;
SEQ ID NO: 31 or at least 95% identity thereof;
SEQ ID NO: 32 or at least 95% identity thereof;
SEQ ID NO: 34 or at least 95% identity thereof;
SEQ ID NO: 35 or at least 95% identity thereof;
SEQ ID NO: 37 or at least 95% identity thereof;
SEQ ID NO: 38 or at least 95% identity thereof;
SEQ ID NO: 40 or at least 95% identity thereof;
SEQ ID NO: 41 or at least 95% identity thereof;
SEQ ID NO: 43 or at least 95% identity thereof;
SEQ ID NO: 44 or at least 95% identity thereof;
SEQ ID NO: 46 or at least 95% identity thereof; or
SEQ ID NO: 47 or at least 95% identity thereof.

6. A method of immunizing a subject, the method comprising:
providing the fusion protein of claim 1; and
administering the fusion protein to the subject in an amount sufficient to induce an immunological response against the fusion protein.

7. The method of claim 6, wherein the oligomerization amino acid sequence includes SEQ ID NO: 1 or at least 95% identity thereof.

8. The method of claim 6, wherein the first spacer amino acid sequence or second spacer amino acid sequence independently includes SEQ ID NO: 2, SEQ ID NO: 49, or SEQ ID NO: 50, or at least 95% identity thereof.

9. The method of claim 6, comprising the purification tag having the purification amino acid sequence of SEQ ID NO: 3 or at least 95% identity thereof.

10. The method of claim 6, wherein the fusion protein comprises:
SEQ ID NO: 4 or at least 95% identity thereof;
SEQ ID NO: 5 or at least 95% identity thereof;
SEQ ID NO: 7 or at least 95% identity thereof;
SEQ ID NO: 8 or at least 95% identity thereof;
SEQ ID NO: 10 or at least 95% identity thereof;
SEQ ID NO: 11 or at least 95% identity thereof;
SEQ ID NO: 13 or at least 95% identity thereof;
SEQ ID NO: 14 or at least 95% identity thereof;
SEQ ID NO: 16 or at least 95% identity thereof;
SEQ ID NO: 17 or at least 95% identity thereof;
SEQ ID NO: 19 or at least 95% identity thereof;
SEQ ID NO: 20 or at least 95% identity thereof;
SEQ ID NO: 22 or at least 95% identity thereof;
SEQ ID NO: 23 or at least 95% identity thereof;
SEQ ID NO: 25 or at least 95% identity thereof;
SEQ ID NO: 26 or at least 95% identity thereof;
SEQ ID NO: 28 or at least 95% identity thereof;
SEQ ID NO: 29 or at least 95% identity thereof;
SEQ ID NO: 31 or at least 95% identity thereof;
SEQ ID NO: 32 or at least 95% identity thereof;
SEQ ID NO: 34 or at least 95% identity thereof;
SEQ ID NO: 35 or at least 95% identity thereof;
SEQ ID NO: 37 or at least 95% identity thereof;
SEQ ID NO: 38 or at least 95% identity thereof;
SEQ ID NO: 40 or at least 95% identity thereof;
SEQ ID NO: 41 or at least 95% identity thereof;
SEQ ID NO: 43 or at least 95% identity thereof;
SEQ ID NO: 44 or at least 95% identity thereof;
SEQ ID NO: 46 or at least 95% identity thereof; or
SEQ ID NO: 47 or at least 95% identity thereof.


SEQUENCE LISTING (50 sequences)

SEQ ID NO: 1 - protein, 95 aa, unidentified
  source 1..95: note = Pseudomonadaceae bacteria
MANEGYHEPIDELTDETRDMHRAIISLMEELEAVDWYNQRVDACKDEALKAILAHNRDEEKEHAAMVLEWIRRKDPRFDKELKDYLFTEKPIAHK

SEQ ID NO: 2 - protein, 7 aa, synthetic construct
GGGSGGG

SEQ ID NO: 3 - protein, 4 aa, synthetic construct
EPEA

SEQ ID NO: 4 - protein, 215 aa, synthetic construct
MANEGYHEPIDELTDETRDMHRAIISLMEELEAVDWYNQRVDACKDEALKAILAHNRDEEKEHAAMVLEWIRRKDPRFDKELKDYLFTEKPIAHKGGGSGGGGMTYTVCEGSKFAWKRPPTDSGHDTVVMEVTYTGSKPCRIPVRAVAHGEPNVNVASLITPNPSMETTGGGFVELQLPPGDNIIYVGELSHQWFQKGSTIGRVLGGSGGGEPEA

SEQ ID NO: 5 - protein, 215 aa, synthetic construct
EPEAGGGSGGGGMTYTVCEGSKFAWKRPPTDSGHDTVVMEVTYTGSKPCRIPVRAVAHGEPNVNVASLITPNPSMETTGGGFVELQLPPGDNIIYVGELSHQWFQKGSTIGRVLGGSGGGMANEGYHEPIDELTDETRDMHRAIISLMEELEAVDWYNQRVDACKDEALKAILAHNRDEEKEHAAMVLEWIRRKDPRFDKELKDYLFTEKPIAHK

SEQ ID NO: 6 - protein, 103 aa, Kyasanur Forest disease virus
GMTYTVCEGSKFAWKRPPTDSGHDTVVMEVTYTGSKPCRIPVRAVAHGEPNVNVASLITPNPSMETTGGGFVELQLPPGDNIIYVGELSHQWFQKGSTIGRVL

SEQ ID NO: 7 - protein, 215 aa, synthetic construct
MANEGYHEPIDELTDETRDMHRAIISLMEELEAVDWYNQRVDACKDEALKAILAHNRDEEKEHAAMVLEWIRRKDPRFDKELKDYLFTEKPIAHKGGGSGGGTTYSMCDKTKFKWKRVPVDSGHDTVVMEVSYTGSDKPCRIPVRAVAHGVPTTNVAMLITPNPTIETSGGGFIEMQLPLGDNIIYVGDLSQQWFQKGSTIGRMFGGSGGGEPEA

SEQ ID NO: 8 - protein, 215 aa, synthetic construct
EPEAGGGSGGGTTYSMCDKTKFKWKRVPVDSGHDTVVMEVSYTGSDKPCRIPVRAVAHGVPTTNVAMLITPNPTIETSGGGFIEMQLPLGDNIIYVGDLSQQWFQKGSTIGRMFGGSGGGMANEGYHEPIDELTDETRDMHRAIISLMEELEAVDWYNQRVDACKDEALKAILAHNRDEEKEHAAMVLEWIRRKDPRFDKELKDYLFTEKPIAHK

SEQ ID NO: 9 - protein, 103 aa, Powassan virus
TTYSMCDKTKFKWKRVPVDSGHDTVVMEVSYTGSDKPCRIPVRAVAHGVPTTNVAMLITPNPTIETSGGGFIEMQLPLGDNIIYVGDLSQQWFQKGSTIGRMF

SEQ ID NO: 10 - protein, 212 aa, synthetic construct
MANEGYHEPIDELTDETRDMHRAIISLMEELEAVDWYNQRVDACKDEALKAILAHNRDEEKEHAAMVLEWIRRKDPRFDKELKDYLFTEKPIAHKGGGSGGGLKGVSYVMCTGSFKLEKEVAETQHGTVLVQVKYEGTDAPCKIPISTQDEKGVTQNGRLITANPIVTDKEKPVNIETEPPFGESYIVIGAGEKALKLSWFKGGSGGGEPEA

SEQ ID NO: 11 - protein, 212 aa, synthetic construct
EPEAGGGSGGGLKGVSYVMCTGSFKLEKEVAETQHGTVLVQVKYEGTDAPCKIPISTQDEKGVTQNGRLITANPIVTDKEKPVNIETEPPFGESYIVIGAGEKALKLSWFKGGSGGGMANEGYHEPIDELTDETRDMHRAIISLMEELEAVDWYNQRVDACKDEALKAILAHNRDEEKEHAAMVLEWIRRKDPRFDKELKDYLFTEKPIAHK

SEQ ID NO: 12 - protein, 100 aa, Dengue virus
  source 1..100: note = Dengue Virus Type 1
LKGVSYVMCTGSFKLEKEVAETQHGTVLVQVKYEGTDAPCKIPISTQDEKGVTQNGRLITANPIVTDKEKPVNIETEPPFGESYIVIGAGEKALKLSWFK

SEQ ID NO: 13 - protein, 214 aa, synthetic construct
MANEGYHEPIDELTDETRDMHRAIISLMEELEAVDWYNQRVDACKDEALKAILAHNRDEEKEHAAMVLEWIRRKDPRFDKELKDYLFTEKPIAHKGGGSGGGQLKGMSYSMCTGKFKVVKEIAETQHGTIVVRVQYEGDGSPCKIPFEIMDLEKRHVLGRLITVNPIVTEKDSPVNIEAEPPFGDSYIIIGVEPGQLKLSWFKKGGSGGGEPEA

SEQ ID NO: 14 - protein, 214 aa, synthetic construct
EPEAGGGSGGGQLKGMSYSMCTGKFKVVKEIAETQHGTIVVRVQYEGDGSPCKIPFEIMDLEKRHVLGRLITVNPIVTEKDSPVNIEAEPPFGDSYIIIGVEPGQLKLSWFKKGGSGGGMANEGYHEPIDELTDETRDMHRAIISLMEELEAVDWYNQRVDACKDEALKAILAHNRDEEKEHAAMVLEWIRRKDPRFDKELKDYLFTEKPIAHK

SEQ ID NO: 15 - protein, 102 aa, Dengue virus
  source 1..102: note = Dengue Virus Type 2
QLKGMSYSMCTGKFKVVKEIAETQHGTIVVRVQYEGDGSPCKIPFEIMDLEKRHVLGRLITVNPIVTEKDSPVNIEAEPPFGDSYIIIGVEPGQLKLSWFKK

SEQ ID NO: 16 - protein, 214 aa, synthetic construct
MANEGYHEPIDELTDETRDMHRAIISLMEELEAVDWYNQRVDACKDEALKAILAHNRDEEKEHAAMVLEWIRRKDPRFDKELKDYLFTEKPIAHKGGGSGGGQLKGMSYSMCTGKFKVVKEIAETQHGTIVIRVQYEGDGSPCKIPFEIMDLEKRHVLGRLITVNPIVTEKDSPVNIEAEPPFGDSYIIIGVDPGQLKLNWFKKGGSGGGEPEA

SEQ ID NO: 17 - protein, 214 aa, synthetic construct
EPEAGGGSGGGQLKGMSYSMCTGKFKVVKEIAETQHGTIVIRVQYEGDGSPCKIPFEIMDLEKRHVLGRLITVNPIVTEKDSPVNIEAEPPFGDSYIIIGVDPGQLKLNWFKKGGSGGGMANEGYHEPIDELTDETRDMHRAIISLMEELEAVDWYNQRVDACKDEALKAILAHNRDEEKEHAAMVLEWIRRKDPRFDKELKDYLFTEKPIAHK

SEQ ID NO: 18 - protein, 102 aa, Dengue virus
  source 1..102: note = Dengue Virus Type 2 (S16803)
QLKGMSYSMCTGKFKVVKEIAETQHGTIVIRVQYEGDGSPCKIPFEIMDLEKRHVLGRLITVNPIVTEKDSPVNIEAEPPFGDSYIIIGVDPGQLKLNWFKK

SEQ ID NO: 19 - protein, 213 aa, synthetic construct
MANEGYHEPIDELTDETRDMHRAIISLMEELEAVDWYNQRVDACKDEALKAILAHNRDEEKEHAAMVLEWIRRKDPRFDKELKDYLFTEKPIAHKGGGSGGGLKGMSYAMCTNTFVLKKEVSETQHGTILIKVEYKGEDAPCKIPFSTEDGQGKAHNGRLITANPVVTKKEEPVNIEAEPPFGESNIVIGIGDNALKINWYKKGGSGGGEPEA

SEQ ID NO: 20 - protein, 213 aa, synthetic construct
EPEAGGGSGGGLKGMSYAMCTNTFVLKKEVSETQHGTILIKVEYKGEDAPCKIPFSTEDGQGKAHNGRLITANPVVTKKEEPVNIEAEPPFGESNIVIGIGDNALKINWYKKGGSGGGMANEGYHEPIDELTDETRDMHRAIISLMEELEAVDWYNQRVDACKDEALKAILAHNRDEEKEHAAMVLEWIRRKDPRFDKELKDYLFTEKPIAHK

SEQ ID NO: 21 - protein, 101 aa, Dengue virus
  source 1..101: note = Dengue Virus Type 3
LKGMSYAMCTNTFVLKKEVSETQHGTILIKVEYKGEDAPCKIPFSTEDGQGKAHNGRLITANPVVTKKEEPVNIEAEPPFGESNIVIGIGDNALKINWYKK

SEQ ID NO: 22 - protein, 212 aa, synthetic construct
MANEGYHEPIDELTDETRDMHRAIISLMEELEAVDWYNQRVDACKDEALKAILAHNRDEEKEHAAMVLEWIRRKDPRFDKELKDYLFTEKPIAHKGGGSGGGIKGMSYTMCSGKFSIDKEMAETQHGTTVVKVKYEGTGAPCKVPIEIRDVNKEKVVGRIISSTPFAENTNSVTNIELEPPFGDSYIVIGVGDSALTLHWFRGGSGGGEPEA

SEQ ID NO: 23 - protein, 212 aa, synthetic construct
EPEAGGGSGGGIKGMSYTMCSGKFSIDKEMAETQHGTTVVKVKYEGTGAPCKVPIEIRDVNKEKVVGRIISSTPFAENTNSVTNIELEPPFGDSYIVIGVGDSALTLHWFRGGSGGGMANEGYHEPIDELTDETRDMHRAIISLMEELEAVDWYNQRVDACKDEALKAILAHNRDEEKEHAAMVLEWIRRKDPRFDKELKDYLFTEKPIAHK

SEQ ID NO: 24 - protein, 100 aa, Dengue virus
  source 1..100: note = Dengue Virus Type 4
IKGMSYTMCSGKFSIDKEMAETQHGTTVVKVKYEGTGAPCKVPIEIRDVNKEKVVGRIISSTPFAENTNSVTNIELEPPFGDSYIVIGVGDSALTLHWFR

SEQ ID NO: 25 - protein, 217 aa, synthetic construct
MANEGYHEPIDELTDETRDMHRAIISLMEELEAVDWYNQRVDACKDEALKAILAHNRDEEKEHAAMVLEWIRRKDPRFDKELKDYLFTEKPIAHKGGGSGGGRLKGVSYSLCTAAFTFTKIPAETLHGTVTVELQYAGTDGPCKVPAQMAVDMQTLTPVGRLITANPVITESTENSKMMLELDPPFGDSYIVIGVGEKKITHHWHRSGGSGGGEPEA

SEQ ID NO: 26 - protein, 217 aa, synthetic construct
EPEAGGGSGGGRLKGVSYSLCTAAFTFTKIPAETLHGTVTVELQYAGTDGPCKVPAQMAVDMQTLTPVGRLITANPVITESTENSKMMLELDPPFGDSYIVIGVGEKKITHHWHRSGGSGGGMANEGYHEPIDELTDETRDMHRAIISLMEELEAVDWYNQRVDACKDEALKAILAHNRDEEKEHAAMVLEWIRRKDPRFDKELKDYLFTEKPIAHK

SEQ ID NO: 27 - protein, 105 aa, Zika virus
RLKGVSYSLCTAAFTFTKIPAETLHGTVTVELQYAGTDGPCKVPAQMAVDMQTLTPVGRLITANPVITESTENSKMMLELDPPFGDSYIVIGVGEKKITHHWHRS

SEQ ID NO: 28 - protein, 314 aa, synthetic construct
MANEGYHEPIDELTDETRDMHRAIISLMEELEAVDWYNQRVDACKDEALKAILAHNRDEEKEHAAMVLEWIRRKDPRFDKELKDYLFTEKPIAHKGGGSGGGLKGTSYKMCTDKMSFVKNPTDTGHGTVVMQVKVPKGAPCKIPVIVADDLTAAINKGILVTVNPIASTNDDEVLIEVNPPFGDSYIIVGTGDSRLTYQWHKEGSSIGKLFTQTMKGAERLAVMGDAAWDFSSAGGFFTSVGKGIHTVFGSAFQGLFGGLSWITKVIMGAVLIWVGINTRNMTMSMSMILVGVIMMFLSLGVGAGGSGGGEPEA

SEQ ID NO: 29 - protein, 314 aa, synthetic construct
EPEAGGGSGGGLKGTSYKMCTDKMSFVKNPTDTGHGTVVMQVKVPKGAPCKIPVIVADDLTAAINKGILVTVNPIASTNDDEVLIEVNPPFGDSYIIVGTGDSRLTYQWHKEGSSIGKLFTQTMKGAERLAVMGDAAWDFSSAGGFFTSVGKGIHTVFGSAFQGLFGGLSWITKVIMGAVLIWVGINTRNMTMSMSMILVGVIMMFLSLGVGAGGSGGGMANEGYHEPIDELTDETRDMHRAIISLMEELEAVDWYNQRVDACKDEALKAILAHNRDEEKEHAAMVLEWIRRKDPRFDKELKDYLFTEKPIAHK

SEQ ID NO: 30 - protein, 202 aa, Yellow fever virus
LKGTSYKMCTDKMSFVKNPTDTGHGTVVMQVKVPKGAPCKIPVIVADDLTAAINKGILVTVNPIASTNDDEVLIEVNPPFGDSYIIVGTGDSRLTYQWHKEGSSIGKLFTQTMKGAERLAVMGDAAWDFSSAGGFFTSVGKGIHTVFGSAFQGLFGGLSWITKVIMGAVLIWVGINTRNMTMSMSMILVGVIMMFLSLGVGA

SEQ ID NO: 31 - protein, 219 aa, synthetic construct
MANEGYHEPIDELTDETRDMHRAIISLMEELEAVDWYNQRVDACKDEALKAILAHNRDEEKEHAAMVLEWIRRKDPRFDKELKDYLFTEKPIAHKGGGSGGGLKGTTYGMCTEKFSFAKNPADTGHGTVVIELSYSGSDGPCKIPIVSVASLNDMTPVGRLVTVNPFVATSSANSKVLVEMEPPFGDSYIVVGMGDKQINHHWHKAGSTGGSGGGEPEA

SEQ ID NO: 32 - protein, 219 aa, synthetic construct
EPEAGGGSGGGLKGTTYGMCTEKFSFAKNPADTGHGTVVIELSYSGSDGPCKIPIVSVASLNDMTPVGRLVTVNPFVATSSANSKVLVEMEPPFGDSYIVVGMGDKQINHHWHKAGSTGGSGGGMANEGYHEPIDELTDETRDMHRAIISLMEELEAVDWYNQRVDACKDEALKAILAHNRDEEKEHAAMVLEWIRRKDPRFDKELKDYLFTEKPIAHK

SEQ ID NO: 33 - protein, 107 aa, Japanese encephalitis virus
  source 1..107: note = Japanese B Encephalitis virus
LKGTTYGMCTEKFSFAKNPADTGHGTVVIELSYSGSDGPCKIPIVSVASLNDMTPVGRLVTVNPFVATSSANSKVLVEMEPPFGDSYIVVGMGDKQINHHWHKAGST

SEQ ID NO: 34 - protein, 228 aa, synthetic construct
MANEGYHEPIDELTDETRDMHRAIISLMEELEAVDWYNQRVDACKDEALKAILAHNRDEEKEHAAMVLEWIRRKDPRFDKELKDYLFTEKPIAHKGGGSGGGTTYGVCSKAFKFAGTPADTGHGTVVLELQYTGTDGPCKVPISSVASLNDLTPVGRLVTVNPFVSVATANSKVLIELEPPFGDSYIVVGRGEQQINHHWHKSGSSIGKAFTTTLKGAGGSGGGEPEA

SEQ ID NO: 35 - protein, 228 aa, synthetic construct
EPEAGGGSGGGTTYGVCSKAFKFAGTPADTGHGTVVLELQYTGTDGPCKVPISSVASLNDLTPVGRLVTVNPFVSVATANSKVLIELEPPFGDSYIVVGRGEQQINHHWHKSGSSIGKAFTTTLKGAGGSGGGMANEGYHEPIDELTDETRDMHRAIISLMEELEAVDWYNQRVDACKDEALKAILAHNRDEEKEHAAMVLEWIRRKDPRFDKELKDYLFTEKPIAHK

SEQ ID NO: 36 - protein, 116 aa, West Nile virus
TTYGVCSKAFKFAGTPADTGHGTVVLELQYTGTDGPCKVPISSVASLNDLTPVGRLVTVNPFVSVATANSKVLIELEPPFGDSYIVVGRGEQQINHHWHKSGSSIGKAFTTTLKGA

SEQ ID NO: 37 - protein, 228 aa, synthetic construct
MANEGYHEPIDELTDETRDMHRAIISLMEELEAVDWYNQRVDACKDEALKAILAHNRDEEKEHAAMVLEWIRRKDPRFDKELKDYLFTEKPIAHKGGGSGGGTTYGMCTEKFTFSKNPADTGHGTVVLELQYTGSDGPCKIPISSVASLNDMTPVGRMVTANPYVASSTANAKVLVEIEPPFGDSYIVVGRGDKQINHHWHKEGSSIGKAFSTTLKGAGGSGGGEPEA

SEQ ID NO: 38 - protein, 228 aa, synthetic construct
EPEAGGGSGGGTTYGMCTEKFTFSKNPADTGHGTVVLELQYTGSDGPCKIPISSVASLNDMTPVGRMVTANPYVASSTANAKVLVEIEPPFGDSYIVVGRGDKQINHHWHKEGSSIGKAFSTTLKGAGGSGGGMANEGYHEPIDELTDETRDMHRAIISLMEELEAVDWYNQRVDACKDEALKAILAHNRDEEKEHAAMVLEWIRRKDPRFDKELKDYLFTEKPIAHK

SEQ ID NO: 39 - protein, 116 aa, Murray Valley encephalitis virus
TTYGMCTEKFTFSKNPADTGHGTVVLELQYTGSDGPCKIPISSVASLNDMTPVGRMVTANPYVASSTANAKVLVEIEPPFGDSYIVVGRGDKQINHHWHKEGSSIGKAFSTTLKGA

SEQ ID NO: 40 - protein, 346 aa, synthetic construct
MANEGYHEEISDLSDETRDMHRAIVSLMEELEAVDWYNQRVDAAQDGDLKAILAHNRDEEKEHAAMVLEWIRRKDPAFDKELKDYLFTEKPIAHSTGGGSGGGPGSGDLNGVKPLILKDCSVAGWLLGNPMCDEFIRVPEWSYIVERANPANDLCYPGSLNDYEELKHLLSRINHFEKILIIPKSSWPNHETSLGVSAACPYQGAPSFFRNVVWLIKKNDAYPTIKISYNNTNREDLLILWGIHHSNNAEEQTNLYKNPTTYISVGTSTLNQRLVPKIATRSQVNGQRGRMDFFWTILKPDDAIHFESNGNFIAPEYAYKIVKKGDSTIMKSGVEYGGSGGGEPEA

SEQ ID NO: 41 - protein, 346 aa, synthetic construct
EPEAGGGSGGGPGSGDLNGVKPLILKDCSVAGWLLGNPMCDEFIRVPEWSYIVERANPANDLCYPGSLNDYEELKHLLSRINHFEKILIIPKSSWPNHETSLGVSAACPYQGAPSFFRNVVWLIKKNDAYPTIKISYNNTNREDLLILWGIHHSNNAEEQTNLYKNPTTYISVGTSTLNQRLVPKIATRSQVNGQRGRMDFFWTILKPDDAIHFESNGNFIAPEYAYKIVKKGDSTIMKSGVEYGGSGGGMANEGYHEEISDLSDETRDMHRAIVSLMEELEAVDWYNQRVDAAQDGDLKAILAHNRDEEKEHAAMVLEWIRRKDPAFDKELKDYLFTEKPIAHST

SEQ ID NO: 42 - protein, 229 aa, unidentified
  source 1..229: note = H5N1 Influenza virus (Type H5)
DLNGVKPLILKDCSVAGWLLGNPMCDEFIRVPEWSYIVERANPANDLCYPGSLNDYEELKHLLSRINHFEKILIIPKSSWPNHETSLGVSAACPYQGAPSFFRNVVWLIKKNDAYPTIKISYNNTNREDLLILWGIHHSNNAEEQTNLYKNPTTYISVGTSTLNQRLVPKIATRSQVNGQRGRMDFFWTILKPDDAIHFESNGNFIAPEYAYKIVKKGDSTIMKSGVEY

SEQ ID NO: 43 - protein, 346 aa, synthetic construct
MANEGYHEEISDLSDETRDMHRAIVSLMEELEAVDWYNQRVDAAQDGDLKAILAHNRDEEKEHAAMVLEWIRRKDPAFDKELKDYLFTEKPIAHSTGGGSGGGPGSGDLDGVKPLILRDCSVAGWLLGNPMCDEFINVPEWSYIVEKANPVNDLCYPGDFNDYEELKHLLSRINHFEKIQIIPKSSWSSHEASLGVSSACPYQGKSSFFRNVVWLIKKNSTYPTIKRSYNNTNQEDLLVLWGIHHPNDAAEQTKLYQNPTTYISVGTSTLNQRLVPRIATRSKVNGQSGRMEFFWTILKPNDAINFESNGNFIAPEYAYKIVKKGDSTIMKSELEYGGSGGGEPEA

SEQ ID NO: 44 - protein, 350 aa, synthetic construct
MANEGYHEEISDLSDETRDMHRAIVSLMEELEAVDWYNQRVDAAQDGDLKAILAHNRDEEKEHAAMVLEWIRRKDPAFDKELKDYLFTEKPIAHSTEPEAGGGSGGGPGSGDLDGVKPLILRDCSVAGWLLGNPMCDEFINVPEWSYIVEKANPVNDLCYPGDFNDYEELKHLLSRINHFEKIQIIPKSSWSSHEASLGVSSACPYQGKSSFFRNVVWLIKKNSTYPTIKRSYNNTNQEDLLVLWGIHHPNDAAEQTKLYQNPTTYISVGTSTLNQRLVPRIATRSKVNGQSGRMEFFWTILKPNDAINFESNGNFIAPEYAYKIVKKGDSTIMKSELEYGGSGGGEPEA

SEQ ID NO: 45 - protein, 229 aa, unidentified
  source 1..229: note = H5N1 Vietnam Influenza virus (Type H5)
DLDGVKPLILRDCSVAGWLLGNPMCDEFINVPEWSYIVEKANPVNDLCYPGDFNDYEELKHLLSRINHFEKIQIIPKSSWSSHEASLGVSSACPYQGKSSFFRNVVWLIKKNSTYPTIKRSYNNTNQEDLLVLWGIHHPNDAAEQTKLYQNPTTYISVGTSTLNQRLVPRIATRSKVNGQSGRMEFFWTILKPNDAINFESNGNFIAPEYAYKIVKKGDSTIMKSELEY

SEQ ID NO: 46 - protein, 312 aa, synthetic construct
MANEGYHEPIDELTDETRDMHRAIISLMEELEAVDWYNQRVDACKDEALKAILAHNRDEEKEHAAMVLEWIRRKDPRFDKELKDYLFTEKPIAHKGGGSGGGPGSGNITNLCPFPEVFNATPFASVYAWNRPRISNCVADYSVLYNPAPFFTFKCYGVSPTKLNDLCFTNVYADSFVIRGPEVPQIAPGQTGPIADYNYKLPDNFTGCVIAWNSNPLDSAPAGNYNYPYRLFRKSPLKPFERDISTEIYQAGPKPCNGVPGFNCYPPLQSYGFRPTYGVGHQPYRVVVLSFENGTNGTTVCGPGSGGGEPEA

SEQ ID NO: 47 - protein, 312 aa, synthetic construct
EPEAGGGSGGGPGSGNITNLCPFPEVFNATPFASVYAWNRPRISNCVADYSVLYNPAPFFTFKCYGVSPTKLNDLCFTNVYADSFVIRGPEVPQIAPGQTGPIADYNYKLPDNFTGCVIAWNSNPLDSAPAGNYNYPYRLFRKSPLKPFERDISTEIYQAGPKPCNGVPGFNCYPPLQSYGFRPTYGVGHQPYRVVVLSFENGTNGTTVCGPGSGGGMANEGYHEPIDELTDETRDMHRAIISLMEELEAVDWYNQRVDACKDEALKAILAHNRDEEKEHAAMVLEWIRRKDPRFDKELKDYLFTEKPIAHK

SEQ ID NO: 48 - protein, 197 aa, Severe acute respiratory syndrome-related coronavirus
  source 1..197: note = SARS-CoV-2 virus
NITNLCPFPEVFNATPFASVYAWNRPRISNCVADYSVLYNPAPFFTFKCYGVSPTKLNDLCFTNVYADSFVIRGPEVPQIAPGQTGPIADYNYKLPDNFTGCVIAWNSNPLDSAPAGNYNYPYRLFRKSPLKPFERDISTEIYQAGPKPCNGVPGFNCYPPLQSYGFRPTYGVGHQPYRVVVLSFENGTNGTTVCGP

SEQ ID NO: 49 - protein, 5 aa, synthetic construct
GGSGG

SEQ ID NO: 50 - protein, 6 aa, synthetic construct
GGGSGG